(19) United States
(12) Patent Application Publication
(10) Pub. No.: US 2013/0281396 A1
(43) Pub. Date: Oct. 24, 2013

(54) TREATMENT OF DISEASES BY EPIGENETIC REGULATION

(71) Applicants: Kevin G. McLure, Calgary (CA); Peter R. Young, San Francisco, CA (US)

(72) Inventors: Kevin G. McLure, Calgary (CA); Peter R. Young, San Francisco, CA (US)

(73) Assignee: RVX Therapeutics Inc., Calgary (CA)

(21) Appl. No.: 13/832,437

(22) Filed: Mar. 15, 2013

Related U.S. Application Data
(60) Provisional application No. 61/635,541, filed on Apr. 19, 2012.

(51) Int. Cl. C07D 491/052; A61K 45/06; A61K 31/436

(52) U.S. Cl. CPC C07D 491/052; A61K 31/436; A61K 45/06
USPC 514/43; 514/302; 514/64; 514/171; 514/110; 514/291

(57) ABSTRACT

The present disclosure provides non-naturally occurring polyphenol compounds that inhibit the bromodomain and extra terminal domain (BET) proteins. The disclosed compositions and methods can be used for treatment and prevention of cancer as well as sepsis, including NUT midline carcinoma, Burkitt's Lymphoma, Acute Myelogenous Leukemia, and Multiple Myeloma.

The present disclosure relates to methods of using compounds to target bromodomain and extra terminal domain proteins to treat and prevent cancer, as well as other diseases such as sepsis.

Cancer is a group of diseases caused by dysregulated cell proliferation. Therapeutic approaches aim to decrease the numbers of cancer cells by inhibiting cell replication or by inducing cancer cell differentiation or death, but there is still significant unmet medical need for more efficacious therapeutic agents. Cancer cells accumulate genetic and epigenetic changes that alter cell growth and metabolism in order to promote cell proliferation and increased resistance to programmed cell death, or apoptosis. Some of these changes include inactivation of tumor suppressor genes, activation of oncogenes, as well as modifications of the regulation of chromatin structure. Watson, Cancer Discovery 1:477-480 (2011); Morin et al., Nature 476:298-303 (2011).

Many modifications of histones in chromatin have been characterized, including acetylation at multiple lysines in histones H3 and H4. Peserico and Simone, J. Biomed. Biotechnol. 2011:371832 (2011). Histone acetylation is controlled by acetylases (HATs) as well as deacetylases (HDACs), and small molecule HDAC inhibitors have been developed with cancer as an indication. Hoshino and Matsubara, Surg. Today 40:809-815 (2010). Histone acetylation controls gene expression by recruiting protein complexes that bind directly to acetylated lysine via bromodomains. Sanchez and Zhou, Curr. Opin. Drug Discov. Devel. 12(5):659-665 (2009). One such family, the bromodomain and extra terminal domain (BET) proteins, comprises Brd2, Brd3, Brd4, and BrdT each of which contains two bromodomains in tandem that can independently bind to acetylated lysines. Wu and Chiang, J. Biol. Chem. 282(18):13141-13145 (2007). BET proteins exert some of their effects on transcription by recruiting the positive transcription elongation factor b (p-TEFb), which stimulates transcription elongation by phosphorylating the C-terminal domain of RNA polymerase II and results in increased expression of growth promoting genes, such as, for example, c-Myc and the well established cancer target Aurora B. Filippakopoulos et al., Nature 468:1067-1073 (2010).

Molecules that bind to BET proteins and prevent them from binding to chromatin, inhibit transcription and prevent cell replication, which is useful in cancer therapy and other settings. For example, it has been shown that BET proteins can be displaced from the chromatin by small molecule inhibitors, such as, for example, JQ1, I-BET, and I-BET151, which specifically compete with the acetyl-lysine binding pocket of the BET protein bromodomains thereby preventing transcription elongation of their target genes. Filippakopoulos et al. (2010); Nicodeme et al., Nature 468:1119-1123 (2010); Dawson et al., Nature 478:529-533 (2011).

Inhibition of BET bromodomain-promoter interactions results in a subsequent reduction of myc transcription and protein levels. This results in G₁ arrest and extensive apoptosis in a variety of leukemia and lymphoma cell lines. Mertz et al., PNAS 108(40):16669-16674 (2011). The Myc family of proto-oncogenes (c-myc, l-myc, n-myc) is activated in 25-35% of all human cancers. Vita and Henrickson, Seminars in Cancer Biol. 16:318-330 (2006). Mouse models of cancer driven by overexpression of c-myc demonstrate that transiently inhibiting c-myc expression can cause tumor regression, cell death, and in some cancers such as leukemia, complete disease remission. Soucek et al., Nature 455:679-683 (2008). The absence of a clear ligand-binding domain of c-myc has made the development of an inhibitor a formidable challenge, thus alternative strategies to targeting c-myc transcription must be developed. Delmore et al., Cell 146:904-917 (2011). A mouse model of aggressive human medulloblastoma, in which c-myc is overexpressed, suggests that BET inhibitors may be useful for treating myc-amplified medulloblastoma. Kawauchi et al., Cancer Cell 21:168-180 (2012); Pei et al., Cancer Cell 21:155-167 (2012). Similarly, inhibition of n-myc through RNA interference significantly reduced tumor growth in neuroblastoma mouse models. Jiang et al., Biochem. Biophs. Res. Commun. 410:364-370 (2011). A similar role for l-myc was suggested in small cell lung carcinoma cell lines using antisense oligonucleotides to inhibit l-myc amplification. Dosaka-Akita et al., Cancer Res. 55:1559-1564 (1995). Therefore BET inhibitors have potential to be efficacious in treating multiple types of cancer.

In fact, small molecules that target the bromodomains of BET family members have demonstrated potential therapeutic use in treating cancer. See, for example, Dawson et al. (2011), showing that a small molecule inhibitor of the BET family has a profound efficacy against human and murine mixed lineage leukemia (MLL)-fusion cell lines by early cell cycle arrest and apoptosis. Its mechanism of efficacy is the selective abrogation of Brd3/4 recruitment to chromatin. BET inhibitor JQ1 has demonstrated potent antitumor activity in murine xenograoft models of NUT (nuclear protein in testis) midline carcinoma (NMC), a rare but lethal form of cancer. NMC tumor cell growth is driven by a translocation of the Brd4 gene to the nutlin 1 gene. Filippakopoulos et al., (2010). JQ1 was also shown to be a potent antiproliferator in multiple myeloma, associated with cell cycle arrest and cellular senescence. Delmore et al. (2011).

BET inhibitors are also expected to be potential therapeutics for other types of cancer. For example, in acute myeloid leukemia (AML), Brd4 is required to sustain myc expression and continued disease progression. Zuber et al., Nature 478:524-8 (2011). Moreover, inactivation of Brd4 results in a rapid and drastic down-regulation of the transcription of the proto-oncogenes c-myc and n-myc in cell lines they are amplified. Dawson et al. (2011); Delmore et al. (2011); Zuber et al. (2011); Mertz et al. (2011). Consequently, treatment of tumors that have activation of c-myc with a BET inhibitor resulted in tumor regression through inactivation of c-myc transcription. BET inhibitors are also expected to have application in multiple myeloma, as the multiple myeloma SET domain (MMSET) which is implicated in this disease also binds to BET proteins. Dawson et al. (2011).

In addition to cancer, BET inhibitors are also expected to have have anti-inflammatory and immunomodulatory properties. Lamotte et al., Bioorganic & Med. Chem. Letters (Feb. 24, 2012); Prinjha et al., Trends Pharmacol. Sci. 33(3):146-153 (2012). BET inhibitors I-BET and I-BET151 decrease IL-6 expression in vivo. I-BET was shown to confer protection against lipopolysaccharide-induced endotoxic shock and bacteria-induced sepsis and I-BET151 was shown to suppress bacterial-induced inflammation and sepsis in a murine model. Nicodeme et al. (2010); Lamotte et al. (2012). In addition, BET inhibitors may modulate responses to viral and bacterial infections, including HIV, herpes, and papilloma viruses.

### DETAILED DESCRIPTION

The present invention provides methods of treating and/or preventing cancer and other diseases by administering a compound that inhibits BET family proteins. Cancers that may be treated or prevented with the methods of the invention include cancers that are sensitive to a compound that binds to bromodomains of BET family proteins, including NUT midline carcinoma, as well as cancers that exhibit c-myc overexpression, including, but not limited to, Burkitt's lymphoma, acute myelogenous leukemia, multiple myeloma, aggressive human medulloblastoma; cancers overexpressing n-myc, cancers that rely on the recruitment of p-TEFb to regulate activated oncogenes such as, for example, NOTCH1. In some embodiments, BET inhibitors may induce apoptosis in cancer cells by decreasing expression of the anti-apoptosis gene Bcl2. In certain embodiments, the methods of the invention are used to treat or prevent cancers, including hematological, epithelial including lung, breast and colon carcinomas, midline carcinomas, mesenchymal, hepatic, renal and neurological tumours.

The methods of the invention include administering to a mammal, such as a human, for the purpose of treating or preventing cancer or other diseases that respond to BET inhibitors, a therapeutically effective amount of one or more compounds selected from the group of compounds of Formula I or a tautomer, stereoisomer, pharmaceutically acceptable salt or hydrate thereof:

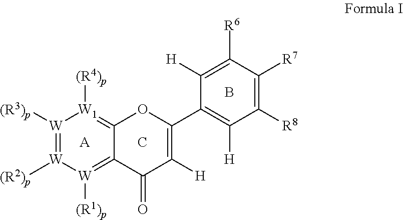

wherein:

R¹, R², R³, R⁴, R⁶, and R⁸ are each independently selected from (C₁-C₈)alkyl, (C₂-C₈)alkenyl, (C₂-C₈)alkynyl, aryl, heteroaryl, alkoxy, aryloxy, arylalkyl, amino, carbonyl, benzyl, phenyl, thioketone, hydrogen, hydroxyl, hydroxyalkyl, aminoalkyl, amides, carbamates, carboxy, cyano, cycloalkyl, ester, ether, formyl, halogen, heterocyclyl, haloalkyl, sulfonic acid [—SO₃H], phosphate, sulfonate, O-glucoronidate [the glucoronic (AKA glucuronic) acid conjugates], dicarboxylic acid, ketone, nitro, sulfide, sulfinyl, sulfonyl, sulfonamide and #STR55#, #STR66#, #STR77#, #STR88#, #STR99#, #STR100#,

R⁷ is selected from alkoxy, hydroxyl, hydroxyalkyl, ether, and ester, or

two adjacent substituents selected from R¹, R², R³, R⁶, R⁷, and R⁸ are connected in a 5 or 6-membered ring to form a bicyclic aryl or bicyclic heteroaryl when W₁ is N;

each W and/or W¹ is independently selected from C and N, wherein if W and/or W¹ is N, then p is 0 and if W and/or W¹ is C, then p is 1;

at least one W and/or W¹ is N;

wherein

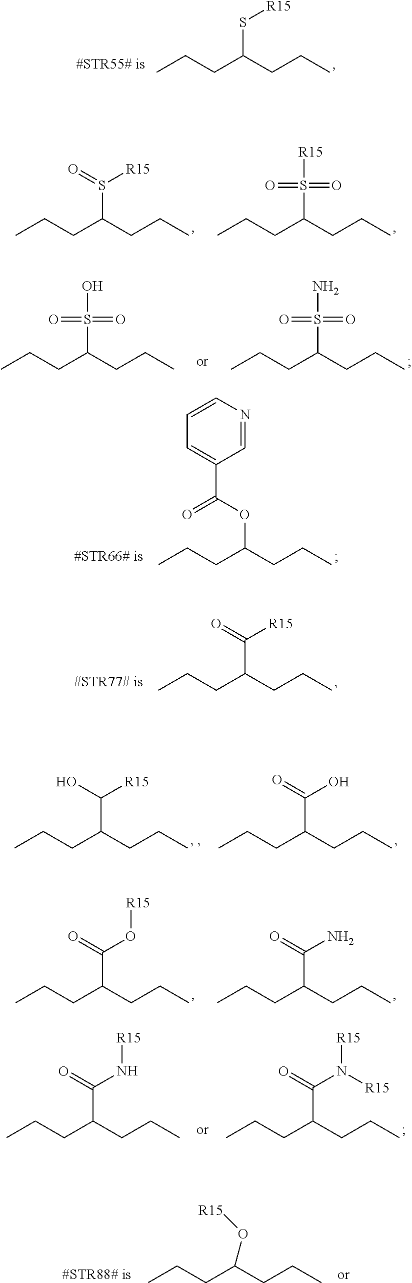

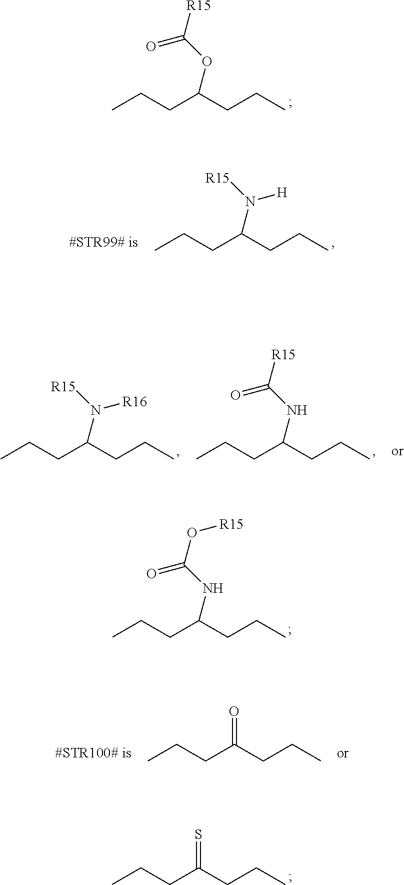

R¹⁵ and R¹⁶ are substituents independently selected from the group consisting of (C₁-C₈)alkyl, (C₂-C₈)alkenyl, (C₂-C₈)alkynyl, aryl, heteroaryl, alkoxy, aryloxy, benzyl, phenyl, carbonyl, hydrogen, hydroxyl [OH], acetyl, hydroxyalkyl, aminoalkyl, amides, carbamates, halogen, CF₃, CCl₃, sulfonic acid [—SO₃H], phosphate, or a derivative thereof, wherein said derivative is optionally substituted and optionally branched, and may have one or more of the C atoms replaced by S, N or O.

In an alternate embodiment, the methods of the invention also include administering to a mammal, such as a human, for the purpose of treating or preventing cancer or other diseases that respond to BET inhibitors, a therapeutically effective amount of one or more compounds selected the group consisting of:
2-(4-Hydroxy-phenyl)-pyrano[2,3-b]pyridin-4-one
2-(4-Hydroxy-phenyl)-pyrano[3,2-b]pyridin-4-one
2-(4-Hydroxyphenyl)-pyrano[2,3-c]pyridin-4-one
2-(3-Fluoro-4-hydroxyphenyl)pyrano[2,3-b]pyridine-4-one
2-(4-Hydroxy-3-methylphenyl)-4H-pyrano[2,3-b]pyridine-4-one
2-(4-Hydroxyphenyl)-4H-pyrano[3,2-c]pyridin-4-one
2-(3-Chloro-4-hydroxyphenyl)-4H-pyrano[2,3-b]pyridine-4-one
2-(3-Bromo-4-hydroxyphenyl)-4H-pyrano[2,3-b]pyridin-4-one
2-(4-Hydroxy-3-methoxyphenyl)-4H-pyrano[2,3-b]pyridine-4-one
2-(4-Methoxyphenyl)-4H-pyrano[2,3-b]pyridine-4-one
2-(4-(2-Hydroxyethoxy)phenyl)-4H-pyrano[2,3-b]pyridine-4-one
4-(4-Oxo-4H-pyrano[2,3-b]pyridine-2-yl)phenyl acetate
2-(4-Hydroxy-3-(hydroxymethyl)phenyl)-4H-pyrano[2,3]-b]pyridine-4-one

and tautomesr, stereoisomers, pharmaceutically acceptable salts, and hydrates thereof.

In certain embodiments, the methods of the invention are useful for the prevention or treatment of diseases that benefit from increased cell death or differentiation, or decreased cell proliferation. This may occur by, for example, decreased expression of a Myc family member or an oncogene required for tumor growth, or increase of a tumor suppressor gene, the latter antagonized by BET proteins. The method of the invention can be used to increase cancer cell death or decrease cancer cell proliferation, including, for example, by decreasing expression of Myc family member. Decreasing expression of the Myc family member may refer to, but is not limited to, transcriptionally modulating the expression of its gene or genes that have been either amplified in the genome or translocated to another chromosomal location, or transcriptionally altered in order to increase its expression (i.e. overexpression) thereby affecting the level of the c-myc protein produced. A decrease in the Myc family member mRNA levels may decrease proliferation of cancer cells and/or increase cancer cell death, including but not limited to apoptosis.

In other embodiments, the methods of the invention are useful for the prevention or treatment of diseases such as cancer in combination with other drugs. In some embodiments, any one of a compound of Formula I may be administered in combination with a standard of care drug(s) for any given tumor type, including, but not limited to, bortezomib, thalidomide, dexamethasone, 5-azacitidine, decitabine, vorinostat, or cyclophosphamide in multiple myeloma. In another embodiment, a compound selected from Formula I may be administered in combination with a PI3K or mTOR inhibitor such as rapamycin or a rapamycin analog. Similarly, a compound selected from Formula I could be administered in combination with gamma secretase inhibitors which inhibit NOTCH1 (given the relationship between c-myc and NOTCH1) or AMPK inducers such as metformin or phenformin for leukemia. Another example of a potentially useful combination is combining a BET inhibitor which decreases myc expression, with an ornithine decarboxylase inhibitor such as difluoromethylornithine that inhibits a myc target.

In certain embodiments, the methods of the invention provide treatment of autoimmune and inflammatory diseases or conditions by administering a compound disclosed herein. In other embodiments, the compounds disclosed herein for use in the methods of the invention may be employed to treat diseases or conditions caused by bacterial or viral infection, such as, for example, infection by HIV, HPV, or herpes virus. In certain embodiments of the invention, one or more of the compounds disclosed herein may be used in the manufacture of a medicament for the treatment of cancer, autoimmune disease, inflammatory disease, AIDS, or sepsis.

### DEFINITIONS

As used in the present specification, the following words, phrases and symbols are generally intended to have the meanings as set forth below, except to the extent that the context in which they are used indicates otherwise. The following abbreviations and terms have the indicated meanings throughout.

“Subject” refers to an animal, such as a mammal, that has been or will be the object of treatment, observation, or experiment. The methods described herein may be useful for both human therapy and veterinary applications. In one embodiment, the subject is a human.

As used herein, “treatment” or “treating” refers to an amelioration of a disease or disorder, or at least one discernible symptom thereof. In another embodiment, “treatment” or “treating” refers to an amelioration of at least one measurable physical parameter, not necessarily discernible by the patient. In yet another embodiment, “treatment” or “treating” refers to inhibiting the progression of a disease or disorder, either physically, for example, stabilization of a discernible symptom, physiologically, for example, stabilization of a physical parameter, or both. In yet another embodiment, “treatment” or “treating” refers to delaying the onset of a disease or disorder.

As used herein, “inhibiting” refers to blocking, suppressing, or in any other way, reducing, the biological function of a BETprotein in a subject.

As used herein, “reducing” refers to reducing the overall levels of BET biological activity, for example, by inhibiting the availability of the level of BET protein in the body for other biological interactions.

The term “autoimmune and inflammatory diseases or conditions” as used herein refers to a wide variety of chronic autoimmune and inflammatory conditions such as rheumatoid arthritis, osteoarthritis, acute gout, psoriasis, systemic lupus erythematosus, multiple sclerosis, inflammatory bowel disease (Crohn's disease and Ulcerative colitis), dry eye, asthma, chronic obstructive airways disease, pneumonitis, myocarditis, pericarditis, myositis, eczema, dermatitis, alopecia, vitiligo, bullous skin diseases, nephritis, vasculitis, atherosclerosis, Alzheimer's disease, Celiac disease, depression, retinitis, uveitis, scleritis, hepatitis, pancreatitis, primary biliary cirrhosis, sclerosing cholangitis, Addison's disease, hypophysitis, thyroiditis, type I diabetes and acute rejection of transplanted organs.

The term “autoimmune and inflammatory diseases or conditions” is also intended to include acute inflammatory conditions such as acute gout, giant cell arteritis, nephritis including lupus nephritis, vasculitis with organ involvement such as glomerulonephritis, vasculitis including giant cell arteritis, Wegener's granulomatosis, Polyarteritis nodosa, Behcet's disease, Kawasaki disease, Takayasu's Arteritis, vasculitis with organ involvement and acute rejection of transplanted organs. The term “autoimmune and inflammatory diseases or conditions” is also intended to include diseases or conditions which involve inflammatory responses to infections with bacteria, viruses, fungi, parasites or their toxins, such as sepsis, sepsis syndrome, septic shock, endotoxaemia, systemic inflammatory response syndrome (SIRS), multi-organ dysfunction syndrome, toxic shock syndrome, acute lung injury, ARDS (adult respiratory distress syndrome), acute renal failure, fulminant hepatitis, burns, acute pancreatitis, postsurgical syndromes, sarcoidosis, Herxheimer reactions, encephalitis, myelitis, meningitis, malaria, and SIRS associated with viral infections such as influenza, herpes zoster, herpes simplex, and coronavirus.

As used herein, the term “effective amount” means that amount of a compound of Formula I or a tautomer, stereoisomer, pharmaceutically acceptable salt, or hydrate thereof, that will elicit the biological or medical response of a tissue, system, animal or human that is being sought, for instance, by a researcher or clinician. Furthermore, the term “therapeutically effective amount” means any amount which, as compared to a corresponding subject who has not received such amount, results in improved treatment, healing, prevention, or amelioration of a disease, disorder, or side effect, or a decrease in the rate of advancement of a disease or disorder. The term also includes within its scope amounts effective to enhance normal physiological function.

As used herein, “prevention” or “preventing” refers to a reduction of the risk of acquiring a given disease or disorder.

A dash (“-”) that is not between two letters or symbols is used to indicate a point of attachment for a substituent. For example, —CONH₂ is attached through the carbon atom.

By “optional” or “optionally” is meant that the subsequently described event or circumstance may or may not occur, and that the description includes instances where the event or circumstance occurs and instances in which is does not. For example, “optionally substituted aryl” encompasses both “aryl” and “substituted aryl” as defined below. It will be understood by those skilled in the art, with respect to any group containing one or more substituents, that such groups are not intended to introduce any substitution or substitution patterns that are sterically impractical, synthetically non-feasible and/or inherently unstable.

As used herein, the term “hydrate” refers to a crystal form with either a stoichiometric or non-stoichiometric amount of water is incorporated into the crystal structure.

The term “aldehyde” or “formyl” as used herein refers to the radical —CHO.

The term “alkenyl” as used herein refers to an unsaturated straight or branched hydrocarbon having at least one carbon-carbon double bond, such as a straight or branched group of 2-22, 2-8, or 2-6 carbon atoms, referred to herein as (C₂-C₂₂)alkenyl, (C₂-C₈)alkenyl, and (C₂-C₆)alkenyl, respectively. Exemplary alkenyl groups include, but are not limited to, vinyl, allyl, butenyl, pentenyl, hexenyl, butadienyl, pentadienyl, hexadienyl, 2-ethylhexenyl, 2-propyl-2-butenyl, 4-(2-methyl-3-butene)-pentenyl, etc.

The term “alkoxy” as used herein refers to an alkyl group attached to an oxygen (—O-alkyl-). “Alkoxy” groups also include an alkenyl group attached to an oxygen (“alkenoxy”) or an alkynyl group attached to an oxygen (“alkynoxy”) groups. Exemplary alkoxy groups include, but are not limited to, groups with an alkyl, alkenyl or alkynyl group of 1-22, 1-8, or 1-6 carbon atoms, referred to herein as (C₁-C₂₂)alkoxy, (C₁-C₈)alkoxy, and (C₁-C₆)alkoxy, respectively. Exemplary alkoxy groups include, but are not limited to methoxy, ethoxy, etc.

The term “alkyl” as used herein refers to a saturated straight or branched hydrocarbon, such as a straight or branched group of 1-22, 1-8, or 1-6 carbon atoms, referred to herein as (C₁-C₂₂)alkyl, (C₁-C₈)alkyl, and (C₁-C₆)alkyl, respectively. Exemplary alkyl groups include, but are not limited to, methyl, ethyl, propyl, isopropyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, butyl, isobutyl, t-butyl, pentyl, isopentyl, neopentyl, hexyl, heptyl, octyl, etc.

The term “alkynyl” as used herein refers to an unsaturated straight or branched hydrocarbon having at least one carbon-carbon triple bond, such as a straight or branched group of 2-22, 2-8, or 2-6 carbon atoms, referred to herein as (C₂-C₂₂)alkynyl, (C₂-C₈)alkynyl, and (C₂-C₆)alkynyl, respectively. Exemplary alkynyl groups include, but are not limited to, ethynyl, propynyl, butynyl, pentynyl, hexynyl, methylpropynyl, 4-methyl-1-butynyl, 4-propyl-2-pentynyl, and 4-butyl-2-hexynyl, etc.

The term “amide” as used herein refers to a radical of the form —RₐC(O)N(R_{b})—, —RₐC(O)N(R_{b})R_{c}—, or —C(O)NR_{b}R_{c}, wherein R_{b} and R_{c} are each independently selected from alkoxy, aryloxy, alkyl, alkenyl, alkynyl, amide, amino, aryl, arylalkyl, carbamate, carboxy, cyano, cycloalkyl, ester, ether, formyl, halogen, haloalkyl, heteroaryl, heterocyclyl, hydrogen, hydroxyl, ketone, nitro, phosphate, sulfide, sulfinyl, sulfonyl, sulfonic acid, sulfonamide and thioketone. The amide can be attached to another group through the carbon, the nitrogen, R_{b}, R_{c}, or Rₐ. The amide also may be cyclic, for example R_{b} and R_{c}, Rₐ and R_{b}, or Rₐ and R_{c} may be joined to form a 3- to 12-membered ring, such as a 3- to 10-membered ring or a 5- to 6-membered ring. The term “amide” encompasses groups such as sulfonamide, urea, carbamate, carbamic acid, and cyclic versions thereof. The term “amide” also encompasses an amide group attached to a carboxy group, for example, -amide-COOH or salts such as -amide-COONa, etc, an amino group attached to a carboxy group, for example, -amino-COOH or salts such as -amino-COONa, etc.

The term “amine” or “amino” as used herein refers to a radical of the form —NR_{d}Rₑ, —N(R_{d})Rₑ—, or —RₑN(R_{d})R_{f}— where R_{d}, Rₑ, and R_{f} are independently selected from alkoxy, aryloxy, alkyl, alkenyl, alkynyl, amide, amino, aryl, arylalkyl, carbamate, carboxy, cyano, cycloalkyl, ester, ether, formyl, halogen, haloalkyl, heteroaryl, heterocyclyl, hydrogen, hydroxyl, ketone, nitro, phosphate, sulfide, sulfinyl, sulfonyl, sulfonic acid, sulfonamide and thioketone. The amino can be attached to the parent molecular group through the nitrogen, R_{d}, Rₑ or R_{f}. The amino also may be cyclic, for example any two of Rₐ, R_{b}, and

R_{c} may be joined together or with the N to form a 3- to 12-membered ring, for example, morpholino or piperidinyl. The term amino also includes the corresponding quaternary ammonium salt of any amino group, for example, —[N(R_{d})(Rₑ)(R_{f})]⁺. Exemplary amino groups include aminoalkyl groups, wherein at least one of R_{d}, Rₑ, or R_{f} is an alkyl group.

The term “aryl” as used herein refers to a mono-, bi-, or other multi-carbocyclic, aromatic ring system. The aryl group can optionally be fused to one or more rings selected from aryls, cycloalkyls, and heterocyclyls. The aryl groups of this invention can be substituted with groups selected from alkoxy, aryloxy, alkyl, alkenyl, alkynyl, amide, amino, aryl, arylalkyl, carbamate, carboxy, cyano, cycloalkyl, ester, ether, formyl, halogen, haloalkyl, heteroaryl, heterocyclyl, hydroxyl, ketone, nitro, phosphate, sulfide, sulfinyl, sulfonyl, sulfonic acid, sulfonamide and thioketone. Exemplary aryl groups include, but are not limited to, phenyl, tolyl, anthracenyl, fluorenyl, indenyl, azulenyl, and naphthyl, as well as benzo-fused carbocyclic moieties such as 5,6,7,8-tetrahydronaphthyl. Exemplary aryl groups also include, but are not limited to a monocyclic aromatic ring system, wherein the ring comprises 6 carbon atoms, referred to herein as “(C₆)aryl.”

The term “arylalkyl” as used herein refers to an aryl group having at least one alkyl substituent, for example -aryl-alkyl-. Exemplary arylalkyl groups include, but are not limited to, arylalkyls having a monocyclic aromatic ring system, wherein the ring comprises 6 carbon atoms, referred to herein as “(C₆)arylalkyl.”

The term “aryloxy” as used herein refers to an aryl group attached to an oxygen atom. Exemplary aryloxy groups include, but are not limited to, aryloxys having a monocyclic aromatic ring system, wherein the ring comprises 6 carbon atoms, referred to herein as “(C₆)aryloxy.”

The term “benzyl” as used herein refers to the group —CH₂-phenyl.

The term “bicyclic aryl” as used herein refers to an aryl group fused to another aromatic or non-aromatic carbocylic or heterocyclic ring. Exemplary bicyclic aryl groups include, but are not limited to, naphthyl or partly reduced forms thereof, such as di-, tetra-, or hexahydronaphthyl.

The term “bicyclic heteroaryl” as used herein refers to a heteroaryl group fused to another aromatic or non-aromatic carbocylic or heterocyclic ring. Exemplary bicyclic heteroaryls include, but are not limited to, 5, 6 or 6,6-fused systems wherein one or both rings contain heteroatoms. The term “bicyclic heteroaryl” also encompasses reduced or partly reduced forms of fused aromatic system wherein one or both rings contain ring heteroatoms. The ring system may contain up to three heteroatoms, independently selected from oxygen, nitrogen, or sulfur. The bicyclic system may be optionally substituted with one or more groups selected from alkoxy, aryloxy, alkyl, alkenyl, alkynyl, amide, amino, aryl, arylalkyl, carbamate, carboxy, cyano, cycloalkyl, ester, ether, formyl, halogen, haloalkyl, heteroaryl, heterocyclyl, hydroxyl, ketone, nitro, phosphate, sulfide, sulfinyl, sulfonyl, sulfonic acid, sulfonamide and thioketone. Exemplary bicyclic heteroaryl's include, but are not limited to, quinazolinyl, benzothiophenyl, benzoxazolyl, benzimidazolyl, benzothiazolyl, benzofuranyl, indolyl, quinolinyl, isoquinolinyl, phthalazinyl, benzotriazolyl, benzopyridinyl, and benzofuranyl.

The term “carbamate” as used herein refers to a radical of the form —R_{g}OC(O)N(Rₕ)—, —R_{g}OC(O)N(Rₕ)Rᵢ—, or —OC(O)NRₕRᵢ, wherein R_{g}, Rₕ and Rᵢ are each independently selected from alkoxy, aryloxy, alkyl, alkenyl, alkynyl, amide, amino, aryl, arylalkyl, carbamate, carboxy, cyano, cycloalkyl, ester, ether, formyl, halogen, haloalkyl, heteroaryl, heterocyclyl, hydrogen, hydroxyl, ketone, nitro, phosphate, sulfide, sulfinyl, sulfonyl, sulfonic acid, sulfonamide and thioketone. Exemplary carbamates include, but are not limited to, arylcarbamates or heteroaryl carbamates, for example wherein at least one of R_{g}, Rₕ and Rᵢ are independently selected from aryl or heteroaryl, such as pyridine, pyridazine, pyrimidine, and pyrazine.

The term “carbonyl” as used herein refers to the radical —C(O)—.

The term “carboxy” as used herein refers to the radical —COOH or its corresponding salts, for example —COONa, etc. The term carboxy also includes “carboxycarbonyl,” for example a carboxy group attached to a carbonyl group, for example, —C(O)—COOH or salts such as —C(O)—COONa, etc.

The term “cyano” as used herein refers to the radical —CN.

The term “cycloalkyl” as used herein refers to a monovalent saturated or unsaturated cyclic, bicyclic, or bridged bicyclic hydrocarbon group of 3-12 carbons, or 3-8 carbons, referred to herein as “(C₃-C₈)cycloalkyl,” derived from a cycloalkane. Exemplary cycloalkyl groups include, but are not limited to, cyclohexanes, cyclohexenes, cyclopentanes, and cyclopentenes. Cycloalkyl groups may be substituted with alkoxy, aryloxy, alkyl, alkenyl, alkynyl, amide, amino, aryl, arylalkyl, carbamate, carboxy, cyano, cycloalkyl, ester, ether, formyl, halogen, haloalkyl, heteroaryl, heterocyclyl, hydroxyl, ketone, nitro, phosphate, sulfide, sulfinyl, sulfonyl, sulfonic acid, sulfonamide and thioketone. Cycloalkyl groups can be fused to other cycloalkyl, aryl, or heterocyclyl groups.

The term “dicarboxylic acid” as used herein refers to a group containing at least two carboxylic acid groups such as saturated and unsaturated hydrocarbon dicarboxylic acids and salts thereof. Exemplary dicarboxylic acids include alkyl dicarboxylic acids. Dicarboxylic acids may be substituted with alkoxy, aryloxy, alkyl, alkenyl, alkynyl, amide, amino, aryl, arylalkyl, carbamate, carboxy, cyano, cycloalkyl, ester, ether, formyl, halogen, haloalkyl, heteroaryl, heterocyclyl, hydrogen, hydroxyl, ketone, nitro, phosphate, sulfide, sulfinyl, sulfonyl, sulfonic acid, sulfonamide and thioketone. Dicarboxylic acids include, but are not limited to succinic acid, glutaric acid, adipic acid, suberic acid, sebacic acid, azelaic acid, maleic acid, phthalic acid, aspartic acid, glutamic acid, malonic acid, fumaric acid, (+)/(−)-malic acid, (+)/(−) tartaric acid, isophthalic acid, and terephthalic acid. Dicarboxylic acids further include carboxylic acid derivatives thereof, such as anhydrides, imides, hydrazides, etc., for example, succinic anhydride, succinimide, etc.

The term “ester” refers to a radical having the structure —C(O)O—, —C(O)O—Rⱼ—, —RₖC(O)O—Rⱼ—, or —RₖC(O)O—, where O is not bound to hydrogen, and Rⱼ and Rₖ can independently be selected from alkoxy, aryloxy, alkyl, alkenyl, alkynyl, amide, amino, aryl, arylalkyl, cycloalkyl, ether, formyl, haloalkyl, halogen, heteroaryl, heterocyclyl, ketone, phosphate, sulfide, sulfinyl, sulfonyl, sulfonic acid and thioketone. Rₖ can be a hydrogen, but Rⱼ, cannot be hydrogen. The ester may be cyclic, for example the carbon atom and Rⱼ, the oxygen atom and Rₖ, or Rⱼ and Rₖ may be joined to form a 3- to 12-membered ring. Exemplary esters include, but are not limited to, alkyl esters wherein at least one of Rj or Rk is alkyl, such as -alkyl-C(O)—O—, —C(O)—O-alkyl-, -alkyl-C(O)—O-alkyl-, etc. Exemplary esters also include aryl or heteoraryl esters, for example wherein at least one of Rj or Rk is a heteroaryl group such as pyridine, pyridazine, pyrmidine and pyrazine, such as a nicotinate ester. Exemplary esters also include reverse esters having the structure —RₖC(O)O—, where the oxygen is bound to the parent molecular group. Exemplary reverse esters include succinate, D-argininate, L-argininate, L-lysinate and D-lysinate. Esters also include carboxylic acid anhydrides and acid halides.

The term “ether” refers to a radical having the structure —RₗO—Rₘ—, where Rₗ and Rₘ can independently be alkyl, alkenyl, alkynyl, aryl, cycloalkyl, heterocyclyl, or ether. The ether can be attached to the parent molecular group through Rₗ or Rₘ. Exemplary ethers include, but are not limited to, alkoxyalkyl and alkoxyaryl groups. Ethers also includes polyethers, for example, where one or both of Rₗ and Rₘ are ethers.

The terms “halo” or “halogen” or “Hal” as used herein refer to F, Cl, Br, or I.

The term “haloalkyl” as used herein refers to an alkyl group substituted with one or more halogen atoms. “Haloalkyls” also encompass alkenyl or alkynyl groups substituted with one or more halogen atoms.

The term “heteroaryl” as used herein refers to a mono-, bi-, or multi-cyclic, aromatic ring system containing one or more heteroatoms, for example 1 to 3 heteroatoms, such as nitrogen, oxygen, and sulfur. Heteroaryls can be substituted with one or more substituents including alkoxy, aryloxy, alkyl, alkenyl, alkynyl, amide, amino, aryl, arylalkyl, carbamate, carboxy, cyano, cycloalkyl, ester, ether, formyl, halogen, haloalkyl, heteroaryl, heterocyclyl, hydroxyl, ketone, nitro, phosphate, sulfide, sulfinyl, sulfonyl, sulfonic acid, sulfonamide and thioketone. Heteroaryls can also be fused to non-aromatic rings. Illustrative examples of heteroaryl groups include, but are not limited to, pyridinyl, pyridazinyl, pyrimidyl, pyrazyl, triazinyl, pyrrolyl, pyrazolyl, imidazolyl, (1,2,3,)- and (1,2,4)-triazolyl, pyrazinyl, pyrimidilyl, tetrazolyl, furyl, thienyl, isoxazolyl, thiazolyl, furyl, phenyl, isoxazolyl, and oxazolyl. Exemplary heteroaryl groups include, but are not limited to, a monocyclic aromatic ring, wherein the ring comprises 2 to 5 carbon atoms and 1 to 3 heteroatoms, referred to herein as “(C₂-C₅)heteroaryl.”

The terms “heterocycle,” “heterocyclyl,” or “heterocyclic” as used herein refer to a saturated or unsaturated 3-, 4-, 5-, 6- or 7-membered ring containing one, two, or three heteroatoms independently selected from nitrogen, oxygen, and sulfur. Heterocycles can be aromatic (heteroaryls) or non-aromatic. Heterocycles can be substituted with one or more substituents including alkoxy, aryloxy, alkyl, alkenyl, alkynyl, amide, amino, aryl, arylalkyl, carbamate, carboxy, cyano, cycloalkyl, ester, ether, formyl, halogen, haloalkyl, heteroaryl, heterocyclyl, hydroxyl, ketone, nitro, phosphate, sulfide, sulfinyl, sulfonyl, sulfonic acid, sulfonamide and thioketone.

Heterocycles also include bicyclic, tricyclic, and tetracyclic groups in which any of the above heterocyclic rings is fused to one or two rings independently selected from aryls, cycloalkyls, and heterocycles. Exemplary heterocycles include acridinyl, benzimidazolyl, benzofuryl, benzothiazolyl, benzothienyl, benzoxazolyl, biotinyl, cinnolinyl, dihydrofuryl, dihydroindolyl, dihydropyranyl, dihydrothienyl, dithiazolyl, furyl, homopiperidinyl, imidazolidinyl, imidazolinyl, imidazolyl, indolyl, isoquinolyl, isothiazolidinyl, isothiazolyl, isoxazolidinyl, isoxazolyl, morpholinyl, oxadiazolyl, oxazolidinyl, oxazolyl, piperazinyl, piperidinyl, pyranyl, pyrazolidinyl, pyrazinyl, pyrazolyl, pyrazolinyl, pyridazinyl, pyridyl, pyrimidinyl, pyrimidyl, pyrrolidinyl, pyrrolidin-2-onyl, pyrrolinyl, pyrrolyl, quinolinyl, quinoxaloyl, tetrahydrofuryl, tetrahydroisoquinolyl, tetrahydropyranyl, tetrahydroquinolyl, tetrazolyl, thiadiazolyl, thiazolidinyl, thiazolyl, thienyl, thiomorpholinyl, thiopyranyl, and triazolyl.

The terms “hydroxy” and “hydroxyl” as used herein refers to the radical —OH.

The term “hydroxyalkyl” as used herein refers to a hydroxy radical attached to an alkyl group.

The term “ketone” as used herein refers to a radical having the structure —C(O)—Rn (such as acetyl, —C(O)CH₃) or —Rₙ—C(O)—Rₒ—. The ketone can be attached to another group through Rₙ or Rₒ. Rₙ or Rₒ can be alkyl, alkenyl, alkynyl, cycloalkyl, heterocyclyl or aryl, or Rₙ or Rₒ can be joined to form a 3- to 12-membered ring.

The term “nitro” as used herein refers to the radical —NO₂.

The term “phenyl” as used herein refers to a 6-membered carbocyclic aromatic ring. The phenyl group can also be fused to a cyclohexane or cyclopentane ring. Phenyl can be substituted with one or more substituents including alkoxy, aryloxy, alkyl, alkenyl, alkynyl, amide, amino, aryl, arylalkyl, carbamate, carboxy, cyano, cycloalkyl, ester, ether, formyl, halogen, haloalkyl, heteroaryl, heterocyclyl, hydroxyl, ketone, nitro, phosphate, sulfide, sulfinyl, sulfonyl, sulfonic acid, sulfonamide and thioketone.

The term “phosphate” as used herein refers to a radical having the structure —OP(O)O₂—, —RₓOP(O)O₂—, —OP(O)O₂R_{y}—, or —RₓOP(O)O₂R_{y}—, wherein Rₓ and R_{y} can be alkyl, alkenyl, alkynyl, alkoxy, amide, amino, aryl, aryloxy, carboxy, cyano, cycloalkyl, ester, ether, halogen, heterocyclyl, hydrogen, hydroxy, ketone, nitro, sulfonate, sulfonyl, and thio.

The term “sulfide” as used herein refers to the radical having the structure R_{z}S—, where R_{z} can be alkoxy, aryloxy, alkyl, alkenyl, alkynyl, amide, amino, aryl, arylalkyl, carbamate, carboxy, cycloalkyl, ester, ether, formyl, haloalkyl, heteroaryl, heterocyclyl, and ketone. The term “alkylsulfide” as used herein refers to an alkyl group attached to a sulfur atom.

The term “sulfinyl” as used herein refers to a radical having the structure —S(O)O—, —RₚS(O)O—, —RₚS(O)OR_{q}—, or —S(O)OR_{q}—, wherein Rₚ and Rₛ can be alkoxy, aryloxy, alkyl, alkenyl, alkynyl, amide, amino, aryl, arylalkyl, cycloalkyl, ester, ether, formyl, halogen, haloalkyl, heteroaryl, heterocyclyl, hydroxyl, ketone, nitro, phosphate, sulfide, sulfonyl, sulfonic acid, sulfonamide and thioketone. Exemplary sulfinyl groups include, but are not limited to, alkylsulfinyls wherein at least one of Rₚ or R_{q} is alkyl, alkenyl or alkynyl.

The term “sulfonamide” as used herein refers to a radical having the structure —(Rᵣ)—N—S(O)₂—Rₛ— or —Rₜ(Rᵣ)—N—S(O)₂—Rₛ, where Rₜ, Rᵣ, and Rₛ can be, for example, hydrogen, alkyl, alkenyl, alkynyl, aryl, cycloalkyl, and heterocyclyl. Exemplary sulfonamides include alkylsulfonamides (for example, where Rₛ is alkyl), arylsulfonamides (for example, where Rₛ is aryl), cycloalkyl sulfonamides (for example, where Rₛ is cycloalkyl), and heterocyclyl sulfonamides (for example, where Rₛ is heterocyclyl), etc.

The term “sulfonate” as used herein refers to the radical —OSO₃⁻. Sulfonate includes salts such as —OSO₃Na, —OSO₃K, etc. and the acid —OSO₃H

The term “sulfonic acid” refers to the radical —SO₃H— and its corresponding salts, for example —SO₃K—, —SO₃Na—.

The term “sulfonyl” as used herein refers to a radical having the structure RᵤSO₂—, where Rᵤ can be alkyl, alkenyl, alkynyl, amino, amide, aryl, cycloalkyl, and heterocyclyl, for example, alkylsulfonyl. The term “alkylsulfonyl” as used herein refers to an alkyl group attached to a sulfonyl group. “Alkylsulfonyl” groups can optionally contain alkenyl or alkynyl groups.

The term “thioketone” refers to a radical having the structure —Rᵥ—C(S)—R_{w}—. The ketone can be attached to another group through Rᵥ or R_{w}. Rᵥ or R_{w} can be alkyl, alkenyl, alkynyl, cycloalkyl, heterocyclyl or aryl, or Rᵥ or R_{w} can be joined to form a 3- to 12-membered ring.

“Alkyl,” “alkenyl,” and “alkynyl” groups, collectively referred to as “saturated and unsaturated hydrocarbons,” can be substituted with or interrupted by at least one group selected from alkoxy, aryloxy, alkyl, alkenyl, alkynyl, amide, amino, aryl, arylalkyl, carbamate, carboxy, cyano, cycloalkyl, ester, ether, formyl, halogen, haloalkyl, heteroaryl, heterocyclyl, hydroxyl, ketone, nitro, phosphate, sulfide, sulfinyl, sulfonyl, sulfonic acid, sulfonamide, thioketone, and N.

As used herein, a “suitable substituent” refers to a group that does not nullify the synthetic or pharmaceutical utility of the compounds of the invention or the intermediates useful for preparing them. Examples of suitable substituents include, but are not limited to: C₁₋₂₂, C₁₋₈, and C₁₋₆ alkyl, alkenyl or alkynyl; C₁₋₆ aryl, C₂₋₆ heteroaryl; C₃₋₇ cycloalkyl; C₁₋₂₂, C₁₋₈, and C₁₋₆ alkoxy; C₆ aryloxy; —CN; —OH; oxo; halo, carboxy; amino, such as —NH(C₁₋₂₂, C₁₋₈, or C₁₋₆ alkyl), —N((C₁₋₂₂, C₁₋₈, and C₁₋₆ alkyl)₂, —NH((C₆)aryl), or —N((C₆)aryl)₂; formyl; ketones, such as —CO(C₁₋₂₂, C₁₋₈, and C₁₋₆ alkyl), —CO((C₆ aryl) esters, such as —CO₂(C₁₋₂₂, C₁₋₈, and C₁₋₆ alkyl) and —CO₂ (C₆ aryl). One of skill in art can readily choose a suitable substituent based on the stability and pharmacological and synthetic activity of the compound of the invention.

The term “pharmaceutically acceptable carrier” as used herein refers to any and all solvents, dispersion media, coatings, isotonic and absorption delaying agents, and the like, that are compatible with pharmaceutical administration. The use of such media and agents for pharmaceutically active substances is well known in the art. The compositions may also contain other active compounds providing supplemental, additional, or enhanced therapeutic functions.

The term “pharmaceutically acceptable composition” as used herein refers to a composition comprising at least one compound as disclosed herein formulated together with one or more pharmaceutically acceptable carriers.

The term “pharmaceutically acceptable prodrugs” as used herein represents those prodrugs of the compounds of the present invention that are, within the scope of sound medical judgment, suitable for use in contact with the tissues of humans and lower animals without undue toxicity, irritation, allergic response, commensurate with a reasonable benefit/risk ratio, and effective for their intended use, as well as the zwitterionic forms, where possible, of the compounds of the invention. A discussion is provided in Higuchi et al., “Pro-drugs as Novel Delivery Systems,” ACS Symposium Series, Vol. 14, and in Roche, E. B., ed. Bioreversible Carriers in Drug Design, American Pharmaceutical Association and Pergamon Press, 1987, both of which are incorporated herein by reference.

The term “pharmaceutically acceptable salt(s)” refers to salts of acidic or basic groups that may be present in compounds used in the present compositions. Compounds included in the present compositions that are basic in nature are capable of forming a wide variety of salts with various inorganic and organic acids. The acids that may be used to prepare pharmaceutically acceptable acid addition salts of such basic compounds are those that form non-toxic acid addition salts, i.e., salts containing pharmacologically acceptable anions, including but not limited to sulfate, citrate, matate, acetate, oxalate, chloride, bromide, iodide, nitrate, sulfate, bisulfate, phosphate, acid phosphate, isonicotinate, acetate, lactate, salicylate, citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate and pamoate (i.e., 1,1′-methylene-bis-(2-hydroxy-3-naphthoate)) salts. Compounds included in the present compositions that include an amino moiety may form pharmaceutically acceptable salts with various amino acids, in addition to the acids mentioned above. Compounds included in the present compositions, that are acidic in nature are capable of forming base salts with various pharmacologically acceptable cations. Examples of such salts include alkali metal or alkaline earth metal salts and, particularly, calcium, magnesium, sodium, lithium, zinc, potassium, and iron salts.

The compounds of the disclosure may contain one or more chiral centers and/or double bonds and, therefore, exist as stereoisomers, such as geometric isomers, enantiomers or diastereomers. The term “stereoisomers” when used herein consist of all geometric isomers, enantiomers or diastereomers. These compounds may be designated by the symbols “R” or “S,” depending on the configuration of substituents around the stereogenic carbon atom. The present invention encompasses various stereoisomers of these compounds and mixtures thereof. Stereoisomers include enantiomers and diastereomers. Mixtures of enantiomers or diastereomers may be designated “(±)” in nomenclature, but the skilled artisan will recognize that a structure may denote a chiral center implicitly.

Individual stereoisomers of compounds of the present invention can be prepared synthetically from commercially available starting materials that contain asymmetric or stereogenic centers, or by preparation of racemic mixtures followed by resolution methods well known to those of ordinary skill in the art. These methods of resolution are exemplified by (1) attachment of a mixture of enantiomers to a chiral auxiliary, separation of the resulting mixture of diastereomers by recrystallization or chromatography and liberation of the optically pure product from the auxiliary, (2) salt formation employing an optically active resolving agent, or (3) direct separation of the mixture of optical enantiomers on chiral chromatographic columns. Stereoisomeric mixtures can also be resolved into their component stereoisomers by well known methods, such as chiral-phase gas chromatography, chiral-phase high performance liquid chromatography, crystallizing the compound as a chiral salt complex, or crystallizing the compound in a chiral solvent. Stereoisomers can also be obtained from stereomerically-pure intermediates, reagents, and catalysts by well known asymmetric synthetic methods.

Geometric isomers can also exist in the compounds of the present invention. The present invention encompasses the various geometric isomers and mixtures thereof resulting from the arrangement of substituents around a carbon-carbon double bond or arrangement of substituents around a carbocyclic ring. Substituents around a carbon-carbon double bond are designated as being in the “Z” or “E” configuration wherein the terms “Z” and “E” are used in accordance with IUPAC standards. Unless otherwise specified, structures depicting double bonds encompass both the E and Z isomers.

Substituents around a carbon-carbon double bond alternatively can be referred to as “cis” or “trans,” where “cis” represents substituents on the same side of the double bond and “trans” represents substituents on opposite sides of the double bond. The arrangement of substituents around a carbocyclic ring are designated as “cis” or “trans.” The term “cis” represents substituents on the same side of the plane of the ring, and the term “trans” represents substituents on opposite sides of the plane of the ring. Mixtures of compounds wherein the substituents are disposed on both the same and opposite sides of plane of the ring are designated “cis/trans.”

The compounds disclosed herein may exist as tautomers and both tautomeric forms are intended to be encompassed by the scope of the invention, even though only one tautomeric structure is depicted. For example, any claim to compound A below is understood to include tautomeric structure B, and vice versa, as well as mixtures thereof.

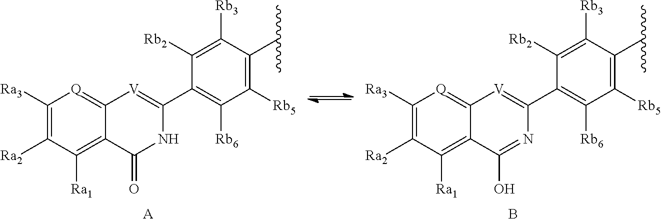

### Embodiments of the Invention

One embodiment of the invention provides methods for inhibiting BET proteins in a mammal comprising administering a therapeutically effective amount of a compound of Formula I or a tautomer, stereoisomer, pharmaceutically acceptable salt, or hydrate thereof:

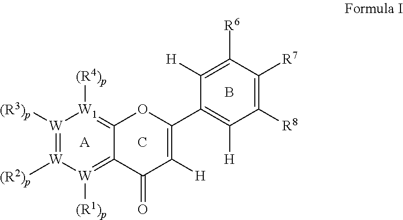

wherein:

R¹, R², R³, R⁴, R⁶, and R⁸ are each independently selected from (C₁-C₈)alkyl, (C₂-C₈)alkenyl, (C₂-C₈)alkynyl, aryl, heteroaryl, alkoxy, aryloxy, arylalkyl, amino, carbonyl, benzyl, phenyl, thioketone, hydrogen, hydroxyl, hydroxyalkyl, aminoalkyl, amides, carbamates, carboxy, cyano, cycloalkyl, ester, ether, formyl, halogen, heterocyclyl, haloalkyl, sulfonic acid [—SO₃H], phosphate, sulfonate, O-glucoronidate [the glucoronic (AKA glucuronic) acid conjugates], dicarboxylic acid, ketone, nitro, sulfide, sulfinyl, sulfonyl, sulfonamide and #STR55#, #STR66#, #STR77#, #STR88#, #STR99#, #STR100#,

R⁷ is selected from alkoxy, hydroxyl, hydroxyalkyl, ether, and ester, or

two adjacent substituents selected from R¹, R², R³, R⁶, R⁷, and R⁸ are connected in a 5- or 6-membered ring to form a bicyclic aryl or bicyclic heteroaryl when W₁ is N;

each W and/or W¹ is independently selected from C and N, wherein if W and/or W¹ is N, then p is 0 and if W and/or W¹ is C, then p is 1;

at least one W and/or W¹ is N;

wherein

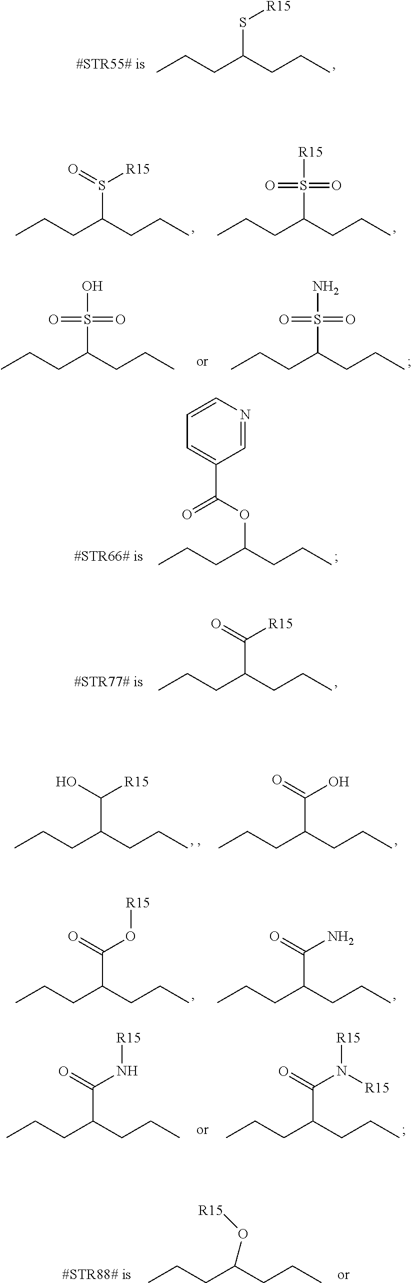

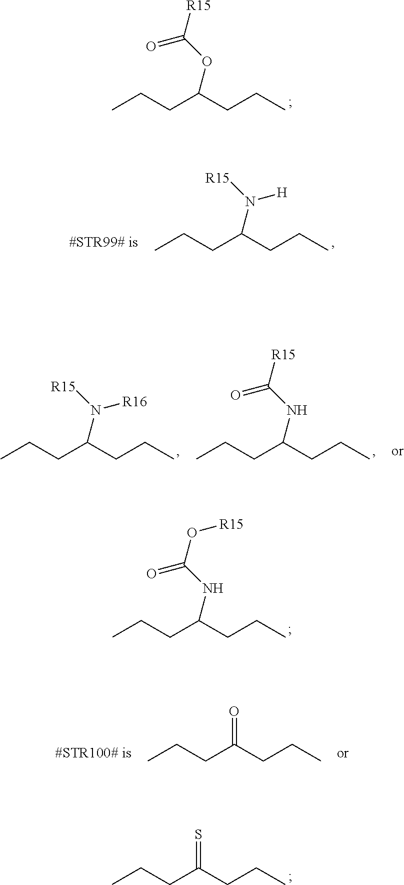

R¹⁵ and R¹⁶ are substituents independently selected from the group consisting of (C₁-C₈)alkyl, (C₂-C₈)alkenyl, (C₂-C₈)alkynyl, aryl, heteroaryl, alkoxy, aryloxy, benzyl, phenyl, carbonyl, hydrogen, hydroxyl [OH], acetyl, hydroxyalkyl, aminoalkyl, amides, carbamates, halogen, CF₃, CCl₃, sulfonic acid [—SO₃H], phosphate, or a derivative thereof, wherein said derivative is optionally substituted and optionally branched, and may have one or more of the C atoms replaced by S, N or O. It will be appreciated by the skilled artisan that when any W and/or W¹ is a nitrogen atom, the nitrogen atom will only bind to three covalent bonds due to available valence electrons.

In some embodiments, the compounds of Formula I have at least one proviso selected from the following:
a. R⁷ is a hydroxyl;
b. at least one W and/or W¹ is a N;
c. at least one of R¹-R⁴, R⁶, or R⁸ is #STR77#, #STR88# or #STR99#;
d. at least one of R¹-R⁴, R⁶, or R⁸ is #STR66#;
e. one of R¹-R⁴ or R⁶-R⁸ is an ester;
f. one of R¹-R⁴, R⁶, or R⁸ is a dicarboxylic acid;
g. one of R¹-R⁴, R⁶, or R⁸ is succinic acid;
h. R² is #STR55#;
i. R⁷ and R² are hydroxyls;
j. at least one of R⁶ and/or R⁸ is different from hydrogen
k. W¹ is nitrogen and R⁷ is hydroxyl and at least one of R⁶ and/or R⁸ is different from hydrogen;
l. R⁷ is a hydroxyl and R² is #STR55#;
m. R⁷ is a hydroxyl and at least one of R⁶ or R⁸ is a halogen;
n. R⁷ is a hydroxyl and at least one W and/or W¹ is a N;
o. R⁷ is a hydroxyl and at least one of R¹-R⁴, R⁶, or R⁸ is #STR66#;
p. R⁷ is a hydroxyl and at least one of R¹-R⁴, R⁶, or R⁸ is #STR77#, #STR88# or #STR99#;
q. at least one of R¹, R², R³, R⁴, R⁶, and R⁸ is a halogen selected from bromide, iodide, fluoride, or chloride;
r. at least one of R¹, R², R³, R⁴, R⁶, and R⁸ is a haloalkyl, such as, but not limited to CF₃ and CCl₃; and
s. at least one of R¹, R², R³, R⁴, R⁶, R⁷, and R⁸ is an ester such as, but not limited to, acetyl.

Another embodiment of the invention provides methods for inhibiting BET proteins in a mammal comprising administering a therapeutically effective amount of a compound selected from the group consisting of:
2-(4-Hydroxy-phenyl)-pyrano[2,3-b]pyridin-4-one
2-(4-Hydroxy-phenyl)-pyrano[3,2-b]pyridin-4-one
2-(4-Hydroxyphenyl)-pyrano[2,3-c]pyridin-4-one
2-(3-Fluoro-4-hydroxyphenyl)pyrano[2,3-b]pyridine-4-one
2-(4-Hydroxy-3-methylphenyl)-4H-pyrano[2,3-b]pyridine-4-one
2-(4-Hydroxyphenyl)-4H-pyrano[3,2-c]pyridin-4-one
2-(3-Chloro-4-hydroxyphenyl)-4H-pyrano[2,3-b]pyridine-4-one
2-(3-Bromo-4-hydroxyphenyl)-4H-pyrano[2,3-b]pyridin-4-one
2-(4-Hydroxy-3-methoxyphenyl)-4H-pyrano[2,3-b]pyridine-4-one
2-(4-Methoxyphenyl)-4H-pyrano[2,3-b]pyridine-4-one
2-(4-(2-Hydroxyethoxy)phenyl)-4H-pyrano[2,3-b]pyridine-4-one
4-(4-Oxo-4H-pyrano[2,3-b]pyridine-2-yl)phenyl acetate
2-(4-Hydroxy-3-(hydroxymethyl)phenyl)-4H-pyrano[2,3]-b]pyridine-4-one

and stereoisomers, tautomers, pharmaceutically acceptable salts, and hydrates thereof.

In one embodiment of the invention, a compound disclosed herein is administered to treat a disease characterized by the involvement of BET proteins. In some embodiments, the disease or condition is cancer. In other embodiments, the disease or condition is selected from diseases associated with systemic inflammatory response syndrome, such as sepsis, burns, pancreatitis, major trauma, haemorrhage and ischaemia. In this embodiment a compound selected from those disclosed herein is administered upon diagnosis to reduce the incidence of SIRS, the onset of shock, multi-organ dysfunction syndrome, which includes the onset of acute lung injury, ARDS, acute renal, hepatic, cardiac and gastrointestinal injury and mortality. In another embodiment, a compound selected from those disclosed herein is administered prior to surgical or other procedures associated with a high risk of sepsis, haemorrhage, extensive tissue damage, SIRS or MODS (multiple organ dysfunction syndrome). In a particular embodiment, a compound selected from those disclosed herein is administered for the treatment of sepsis, sepsis syndrome, septic shock or endotoxaemia. In another embodiment, a compound selected from those disclosed herein is administered for the treatment of acute or chronic pancreatitis.

One embodiment of the invention provides a method for treating or preventing a disease characterized by the involvement of BET proteins in a mammal comprising administering a therapeutically effective amount of a compound of Formula I or a tautomer, stereoisomer, pharmaceutically acceptable salt, or hydrate thereof:

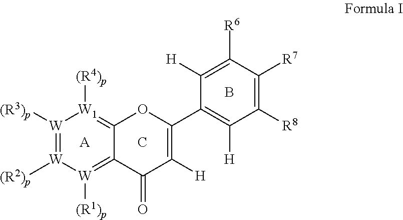

wherein:

R¹, R², R³, R⁴, R⁶, and R⁸ are each independently selected from (C₁-C₃)alkyl, (C₂-C₈)alkenyl, (C₂-C₈)alkynyl, aryl, heteroaryl, alkoxy, aryloxy, arylalkyl, amino, carbonyl, benzyl, phenyl, thioketone, hydrogen, hydroxyl, hydroxyalkyl, aminoalkyl, amides, carbamates, carboxy, cyano, cycloalkyl, ester, ether, formyl, halogen, heterocyclyl, haloalkyl, sulfonic acid [—SO₃H], phosphate, sulfonate, O-glucoronidate [the glucoronic (AKA glucuronic) acid conjugates], dicarboxylic acid, ketone, nitro, sulfide, sulfinyl, sulfonyl, sulfonamide and #STR55#, #STR66#, #STR77#, #STR88#, #STR99#, #STR100#,

R⁷ is selected from alkoxy, hydroxyl, hydroxyalkyl, ether, and ester, or

two adjacent substituents selected from R¹, R², R³, R⁶, R⁷, and R⁸ are connected in a 5 or 6-membered ring to form a bicyclic aryl or bicyclic heteroaryl when W₁ is N;

each W and/or W¹ is independently selected from C and N, wherein if W and/or W¹ is N, then p is 0 and if W and/or W¹ is C, then p is 1;

at least one W and/or W¹ is N;

wherein

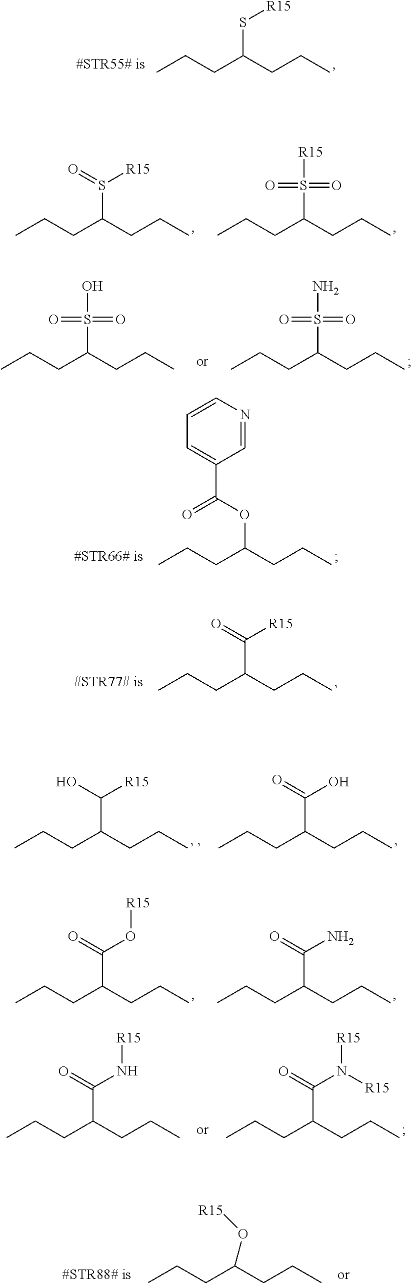

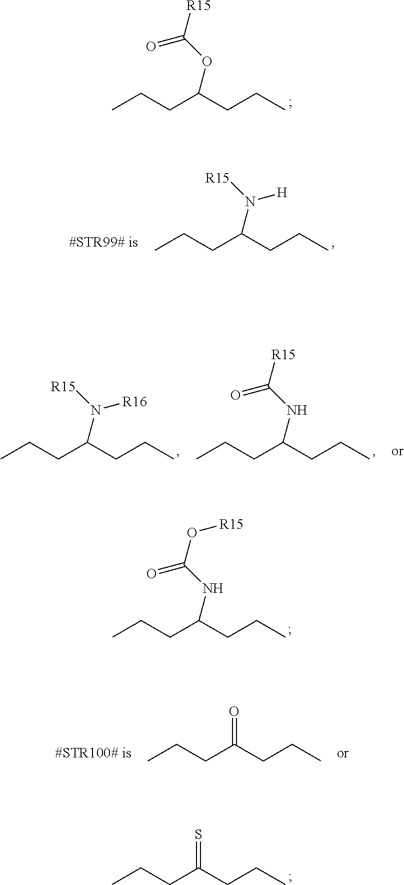

R¹⁵ and R¹⁶ are substituents independently selected from the group consisting of (C₁-C₈)alkyl, (C₂-C₈)alkenyl, (C₂-C₈)alkynyl, aryl, heteroaryl, alkoxy, aryloxy, benzyl, phenyl, carbonyl, hydrogen, hydroxyl [OH], acetyl, hydroxyalkyl, aminoalkyl, amides, carbamates, halogen, CF₃, CCl₃, sulfonic acid [—SO₃H], phosphate, or a derivative thereof, wherein said derivative is optionally substituted and optionally branched, and may have one or more of the C atoms replaced by S, N or O.

In some embodiments, the methods of the invention are carried out by administering a compound of Formula I, wherein at least one of R¹, R², R³, R⁴, R⁶, R⁷, and R⁸ is a nicotinate ester.

In some embodiments, the methods of the invention are carried out by administering a compound of Formula I, wherein R⁷ is hydroxyl, and R⁶ and R⁸ are independently selected from

arylalkyl, carboxy, cyano, cycloalkyl, ester, formyl, halogen, haloalkyl, heteroaryl, heterocyclyl, hydrogen, hydroxyl, hydroxyalkyl, hydroxyaryl, ketone, perfluoroalkyl, perfluorocycloalkyl, O-sulfate, and O-glucoronidate,

subject to the proviso that R⁶ and R⁸ are not both simultaneously hydrogen.

In certain embodiments, a method is provided for treating a disease characterized by the involvement of a BET proteins in a mammal comprising administering a therapeutically effective amount of a compound selected from the group consisting of:
2-(4-Hydroxy-phenyl)-pyrano[2,3-b]pyridin-4-one
2-(4-Hydroxy-phenyl)-pyrano[3,2-b]pyridin-4-one
2-(4-Hydroxyphenyl)-pyrano[2,3-c]pyridin-4-one
2-(3-Fluoro-4-hydroxyphenyl)pyrano[2,3-b]pyridine-4-one
2-(4-Hydroxy-3-methylphenyl)-4H-pyrano[2,3-b]pyridine-4-one
2-(4-Hydroxyphenyl)-4H-pyrano[3,2-c]pyridin-4-one
2-(3-Chloro-4-hydroxyphenyl)-4H-pyrano[2,3-b]pyridine-4-one
2-(3-Bromo-4-hydroxyphenyl)-4H-pyrano[2,3-b]pyridin-4-one
2-(4-Hydroxy-3-methoxyphenyl)-4H-pyrano[2,3-b]pyridine-4-one
2-(4-Methoxyphenyl)-4H-pyrano[2,3-b]pyridine-4-one
2-(4-(2-Hydroxyethoxy)phenyl)-4H-pyrano[2,3-b]pyridine-4-one
4-(4-Oxo-4H-pyrano[2,3-b]pyridine-2-yl)phenyl acetate
2-(4-Hydroxy-3-(hydroxymethyl)phenyl)-4H-pyrano[2,3]-b]pyridine-4-one;

and stereoisomers, tautomers, pharmaceutically acceptable salts, and hydrates thereof.

In some embodiments of the method, the therapeutically effective amount of the compound of Formula I is administered with a pharmaceutically acceptable carrier in a pharmaceutically acceptable composition.

In some embodiments of the method, the therapeutically effective amount of the compound of Formula I is sufficient to establish a concentration ranging from about 0.001 μM to about 100 μM in the mammal.

In some embodiments of the method, the therapeutically effective amount of the compound of Formula I is sufficient to establish a concentration ranging from about 1 μM to about 20 μM.

In some embodiments, the method for inhibiting BET proteins in a mammal further comprises a method of treating or preventing cancer with a therapeutically effective amount of the compound of Formula I.

In some embodiments, the method for inhibiting BET proteins in a mammal further comprises a method of treating or preventing cancer with a therapeutically effective amount of one or more compounds selected from the group consisting of:
2-(4-Hydroxy-phenyl)-pyrano[2,3-b]pyridin-4-one
2-(4-Hydroxy-phenyl)-pyrano[3,2-b]pyridin-4-one
2-(4-Hydroxyphenyl)-pyrano[2,3-c]pyridin-4-one
2-(3-Fluoro-4-hydroxyphenyl)pyrano[2,3-b]pyridine-4-one
2-(4-Hydroxy-3-methylphenyl)-4H-pyrano[2,3-b]pyridine-4-one
2-(4-Hydroxyphenyl)-4H-pyrano[3,2-c]pyridin-4-one
2-(3-Chloro-4-hydroxyphenyl)-4H-pyrano[2,3-b]pyridine-4-one
2-(3-Bromo-4-hydroxyphenyl)-4H-pyrano[2,3-b]pyridin-4-one
2-(4-Hydroxy-3-methoxyphenyl)-4H-pyrano[2,3-b]pyridine-4-one
2-(4-Methoxyphenyl)-4H-pyrano[2,3-b]pyridine-4-one
2-(4-(2-Hydroxyethoxy)phenyl)-4H-pyrano[2,3-b]pyridine-4-one
4-(4-Oxo-4H-pyrano[2,3-b]pyridine-2-yl)phenyl acetate
2-(4-Hydroxy-3-(hydroxymethyl)phenyl)-4H-pyrano[2,3]-b]pyridine-4-one

and stereoisomers, tautomers, pharmaceutically acceptable salts, and hydrates thereof.

In some embodiments of the method, the cancer is selected from midline carcinoma, small cell lung carcinoma, acute myeloid leukemia (AML), mixed lineage leukemia, Burkitt's lymphoma, multiple myeloma, and aggressive human medulloblastoma.

In some embodiments of the method, the cancer involves the recruitment of p-TEFb to regulate activated oncogenes such as, for example, NOTCH 1.

In some embodiments of the method, the cancer involves over-expression of c-myc.

In some embodiments of the method, the cancer involves over-expression of n-myc.

In some embodiments of the method, the cancer is characterized by overexpression of l-myc.

In some embodiments, the method for inhibiting BET proteins in a mammal further comprises a method of treating or preventing cancer with a therapeutically effective amount of the compound of Formula I, and the therapeutically effective amount of a compound disclosed herein is administered in combination with a standard of care drug for any given tumor type, such as for example, but not limited to, bortezomib, thalidomide, dexamethasone, 5-azacitidine, decitabine, vorinostat, or cyclophosphamide in multiple myeloma.

In some embodiments, the method for inhibiting BET proteins in a mammal further comprises a method of treating or preventing cancer with a therapeutically effective amount of the compound of Formula I, and the therapeutically effective amount of the compound disclosed herein is administered in combination with a PI3K or mTOR inhibitor such as rapamycin or BEZ-235 or BKM-120.

In some embodiments, the method for inhibiting BET proteins in a mammal further comprises a method of treating or preventing cancer with a therapeutically effective amount of the compound of Formula I, and the therapeutically effective amount of the compound disclosed herein is administered in combination with gamma secretase inhibitors which inhibit NOTCH1.

In some embodiments, the method for inhibiting BET proteins in a mammal further comprises a method of treating or preventing cancer with a therapeutically effective amount of the compound of Formula I, and the therapeutically effective amount of the compound disclosed herein is administered in combination with AMPK inducers such as metformin or phenformin for treatment of leukemia.

In some embodiments, the method for inhibiting BET proteins in a mammal further comprises a method of treating or preventing cancer with a therapeutically effective amount of 2-(4-Hydroxy-phenyl)-pyrano[2,3-b]pyridin-4-one.

In some embodiments, the method for inhibiting BET proteins in a mammal further comprises a method of treating or preventing cancer with a therapeutically effective amount of 2-(4-Hydroxy-phenyl)-pyrano[3,2-b]pyridin-4-one.

In some embodiments, the method for inhibiting BET proteins in a mammal further comprises a method of treating or preventing cancer with a therapeutically effective amount of 2-(3-Bromo-4-hydroxyphenyl)-4H-pyrano[2,3-b]pyridin-4-one

### Pharmaceutical Formulations and Methods of Treatment

The methods of the invention provide for the administration of pharmaceutical compositions comprising compounds as disclosed herein formulated together with one or more pharmaceutically acceptable carriers. These formulations include those suitable for oral, rectal, topical, intraocular, buccal and parenteral (for example subcutaneous, intramuscular, intradermal, intravenous, or via implants) administration, although the most suitable form of administration in any given case will depend on the degree and severity of the condition being treated and on the nature of the particular compound being used.

Formulations suitable for oral administration may be presented in discrete units, such as capsules, cachets, lozenges, or tablets, each containing a predetermined amount of the compound as powder or granules; as a solution or a suspension in an aqueous or non-aqueous liquid; or as an oil-in-water or water-in-oil emulsion. As indicated, such formulations may be prepared by any suitable method of pharmacy which includes the step of bringing into association the active compound and the carrier or excipient (which may constitute one or more accessory ingredients). The carrier must be acceptable in the sense of being compatible with the other ingredients of the formulation and must not be deleterious to the recipient. The carrier may be a solid or a liquid, or both, and may be formulated with the compound as a unit-dose formulation, for example, a tablet, which may contain from 0.05% to 95% by weight of the active compound. Other pharmacologically active substances may also be present including other compounds. The formulations of the invention may be prepared by any of the well known techniques of pharmacy consisting essentially of admixing the components.

For solid compositions, conventional nontoxic solid carriers include, for example, pharmaceutical grades of mannitol, lactose, starch, magnesium stearate, sodium saccharin, talc, cellulose, glucose, sucrose, magnesium carbonate, and the like. Liquid pharmacologically administrable compositions can, for example, be prepared by dissolving, dispersing, etc., an active compound as described herein and optional pharmaceutical adjuvants in an excipient, such as, for example, water, saline, aqueous dextrose, glycerol, ethanol, and the like, to thereby form a solution or suspension. In general, suitable formulations may be prepared by uniformly and intimately admixing the active compound with a liquid or finely divided solid carrier, or both, and then, if necessary, shaping the product. For example, a tablet may be prepared by compressing or molding a powder or granules of the compound, optionally with one or more assessory ingredients. Compressed tablets may be prepared by compressing, in a suitable machine, the compound in a free-flowing form, such as a powder or granules optionally mixed with a binder, lubricant, inert diluent and/or surface active/dispersing agent(s). Molded tablets may be made by molding, in a suitable machine, the powdered compound moistened with an inert liquid diluent.

Formulations suitable for buccal (sub-lingual) administration include lozenges comprising a compound in a flavored base, usually sucrose and acacia or tragacanth, and pastilles comprising the compound in an inert base such as gelatin and glycerin or sucrose and acacia.

Formulations of the present invention suitable for parenteral administration comprise sterile aqueous preparations of the compounds, which are approximately isotonic with the blood of the intended recipient. These preparations are administered intravenously, although administration may also be effected by means of subcutaneous, intramuscular, or intradermal injection. Such preparations may conveniently be prepared by admixing the compound with water and rendering the resulting solution sterile and isotonic with the blood. Injectable compositions according to the invention may contain from 0.1 to 5% w/w of the active compound.

Formulations suitable for rectal administration are presented as unit-dose suppositories. These may be prepared by admixing the compound with one or more conventional solid carriers, for example, cocoa butter, and then shaping the resulting mixture.

Formulations suitable for topical application to the skin may take the form of an ointment, cream, lotion, paste, gel, spray, aerosol, or oil. Carriers and excipients which may be used include Vaseline, lanoline, polyethylene glycols, alcohols, and combinations of two or more thereof. The active compound is generally present at a concentration of from about 0.1% to about 15% w/w of the composition, for example, from about 0.5 to about 2%.

The amount of active compound administered may be dependent on the subject being treated, the subject's weight, the manner of administration and the judgment of the prescribing physician. For example, a dosing schedule may involve the daily or semi-daily administration of the encapsulated compound at a perceived dosage of about 1 μg to about 1000 mg. In another embodiment, intermittent administration, such as on a monthly or yearly basis, of a dose of the encapsulated compound may be employed. Encapsulation facilitates access to the site of action and allows the administration of the active ingredients simultaneously, in theory producing a synergistic effect. In accordance with standard dosing regimens, physicians will readily determine optimum dosages and will be able to readily modify administration to achieve such dosages.

A therapeutically effective amount of a compound or composition disclosed herein can be measured by the therapeutic effectiveness of the compound. Compounds selected from any of the compounds disclosed herein may be administered in a dose of about 1 μg/kg to about 200 mg/kg daily; such as from about 1 μg/kg to about 150 mg/kg, from about 1 mg/kg to about 200 mg/kg, from about 1 μg/kg to about 100 mg/kg, from about 1 μg/kg to about 1 mg/kg, from about 50 μg/kg to about 200 mg/kg, from about 10 μg/kg to about 1 mg/kg, from about 10 μg/kg to about 100 μg/kg, from about 100 μg to about 10 mg/kg, and from about 500 μg/kg to about 50 mg/kg. The dosages, however, may be varied depending upon the requirements of the patient, the severity of the condition being treated, and the compound being used. In one embodiment, the therapeutically effective amount of a disclosed compound is sufficient to establish a maximal plasma concentration ranging from about 0.001 μM to about 100 μM, for example, from about 1 μM to about 20 μM. Preliminary doses as, for example, determined according to animal tests, and the scaling of dosages for human administration is performed according to art-accepted practices.

Toxicity and therapeutic efficacy can be determined by standard pharmaceutical procedures in cell cultures or experimental animals, for example, for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compositions that exhibit large therapeutic indices are preferable.

The therapeutically effective dose can be estimated initially from cell culture assays. A dose may be formulated in animal models to achieve a circulating plasma concentration range that includes the IC₅₀ (i.e., the concentration of the therapeutic which achieves a half-maximal inhibition of symptoms) as determined in cell culture assays or animal models. Levels in plasma may be measured, for example, by high performance liquid chromatography. The effects of any particular dosage can be monitored by a suitable bioassay. Examples of dosages are: about 0.1×IC₅₀, about 0.5×IC₅₀, about 1×IC₅₀, about 5×IC₅₀, 10×IC₅₀, about 50×IC₅₀, and about 100×IC₅₀.

Data obtained from the cell culture assays or animal studies can be used in formulating a range of dosage for use in humans. Therapeutically effective dosages achieved in one animal model may be converted for use in another animal, including humans, using conversion factors known in the art (see, for example, Freireich et al., Cancer Chemother. Reports 50(4):219-244 (1966) and Table 1 for Equivalent Surface Area Dosage Factors).

TABLE 1

To:

| | Mouse | Rat | Monkey | Dog | Human |
|---|---|---|---|---|---|
| From: | (20 g) | (150 g) | (3.5 kg) | (8 kg) | (60 kg) |
| Mouse | 1 | ½ | ¼ | ⅙ | 1/12 |
| Rat | 2 | 1 | ½ | ¼ | 1/7 |
| Monkey | 4 | 2 | 1 | ⅗ | ⅓ |
| Dog | 6 | 4 | ⅗ | 1 | ½ |
| Human | 12 | 7 | 3 | 2 | 1 |

The dosage of such compounds lies preferably within a range of circulating concentrations that include the ED₅₀ with little or no toxicity. The dosage may vary within this range depending upon the dosage form employed and the route of administration utilized. Generally, a therapeutically effective amount may vary with the subject's age, condition, and sex, as well as the severity of the medical condition in the subject. The dosage may be determined by a physician and adjusted, as necessary, to suit observed effects of the treatment.

In one embodiment, a compound disclosed herein is administered in combination with another therapeutic agent. The other therapeutic agent can provide additive or synergistic value relative to the administration of a compound disclosed herein alone. The therapeutic agent can be, for example, bortezomib, thalidomide, dexamethasone, 5-azacitidine, decitabine, vorinostat, or cyclophosphamide in multiple myeloma, or, for example, an a compound could be administered in combination with a PI3K or mTOR inhibitor such as rapamycin (Muellner et al., Nat. Chem. Biol. 7:787-793 (2011)), or BEZ-235 or BKM-120 (Pei et al. 2012)). Similarly, a compound disclosed herein for use in the methods of the invention could be administered in combination with gamma secretase inhibitors which inhibit NOTCH1 (given the relationship between c-myc and NOTCH1 (Palomero and Ferrando, Clin. Cancer. Res. (2008), Demarest et al., Blood 117:2901-2909 (2011)), or AMPK inducers such as metformin or phenformin which also holds promises for some leukemias (Green et al., Blood 116:4262-4273 (2010), Grimaldi et al., Leukemia 26:91-100 (2012)). Another example of a potentially useful combination is combining a BET inhibitor which decreases myc expression, with an ornithine decarboxylase inhibitor such as difluoromethylornithine that inhibits a myc target (Nilsson et al., Cancer Cell (2005)).

In one embodiment, a method of inhibiting BET proteins comprises administering a therapeutically effective amount of a compound disclosed herein. The compound may be administered as a pharmaceutically acceptable composition, comprising a compound selected from compounds disclosed herein for use in the methods of the invention and a pharmaceutically acceptable carrier. Another embodiment provides a method of treating or preventing cancer or diseases involving BET protein related disorders, comprising administering to a mammal a therapeutically effective amount of a presently a compound disclosed herein or composition comprising such compound.

### EXAMPLES

The compounds disclosed herein for use in the methods of the invention may be prepared according to any method known in the art, including but not limited to, methods disclosed in United States Patent Publication 2009/0259038, especially, paragraphs 1213-1228, incorporated herein by reference.

Examples of compounds of Formula I that may be used in any of the methods of the invention include, but are not limited to, the following compounds:

### a) 2-(4-Hydroxy-phenyl)-pyrano[2,3-b]pyridin-4-one

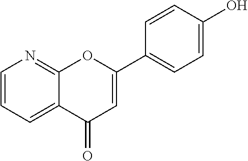 [SMILES: O=c1cc(-c2ccc(O)cc2)oc2ncccc12]

and its derivatives, including but not limited to:
2-(2,4-Dihydroxy-phenyl)-pyrano[2,3-b]pyridin-4-one
2-(3,4-Dihydroxy-phenyl)-pyrano[2,3-b]pyridin-4-one
5-Hydroxy-2-(4-hydroxy-phenyl)-pyrano[2,3-b]pyridin-4-one
6-Hydroxy-2-(4-hydroxy-phenyl)-pyrano[2,3-b]pyridin-4-one
7-Hydroxy-2-(4-hydroxy-phenyl)-pyrano[2,3-b]pyridin-4-one
[5-Hydroxy-2-(4-oxo-4H-pyrano[2,3-b]pyridin-2-yl)-phenyl]-carbamic acid ethyl ester
[3,5-Dihydroxy-2-(4-oxo-4H-pyrano[2,3-b]pyridin-2-yl)-phenyl]-carbamic acid ethyl ester
[4,5-Dihydroxy-2-(4-oxo-4H-pyrano[2,3-b]pyridin-2-yl)-phenyl]-carbamic acid ethyl ester
[5-Hydroxy-2-(5-hydroxy-4-oxo-4H-pyrano[2,3-b]pyridin-2-yl)-phenyl]-carbamic acid ethyl ester
[5-Hydroxy-2-(6-hydroxy-4-oxo-4H-pyrano[2,3-b]pyridin-2-yl)-phenyl]-carbamic acid ethyl ester
[5-Hydroxy-2-(7-hydroxy-4-oxo-4H-pyrano[2,3-b]pyridin-2-yl)-phenyl]-carbamic acid ethyl ester

### b) 2-(4-Hydroxy-phenyl)-pyrano[2,3-c]pyridin-4-one

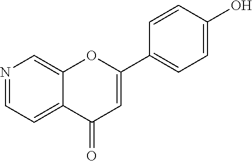 [SMILES: O=c1cc(-c2ccc(O)cc2)oc2cnccc12]

and its derivatives, including but not limited to:
2-(2,4-Dihydroxy-phenyl)-pyrano[2,3-c]pyridin-4-one
2-(3,4-Dihydroxy-phenyl)-pyrano[2,3-c]pyridin-4-one
5-Hydroxy-2-(4-hydroxy-phenyl)-pyrano[2,3-c]pyridin-4-one
6-Hydroxy-2-(4-hydroxy-phenyl)-pyrano[2,3-c]pyridin-4-one
8-Hydroxy-2-(4-hydroxy-phenyl)-pyrano[2,3-c]pyridin-4-one
[5-Hydroxy-2-(4-oxo-4H-pyrano[2,3-c]pyridin-2-yl)-phenyl]-carbamic acid ethyl ester
[3,5-Dihydroxy-2-(4-oxo-4H-pyrano[2,3-c]pyridin-2-yl)-phenyl]-carbamic acid ethyl ester
[4,5-Dihydroxy-2-(4-oxo-4H-pyrano[2,3-c]pyridin-2-yl)-phenyl]-carbamic acid ethyl ester
[5-Hydroxy-2-(5-hydroxy-4-oxo-4H-pyrano[2,3-c]pyridin-2-yl)-phenyl]-carbamic acid ethyl ester
[5-Hydroxy-2-(6-hydroxy-4-oxo-4H-pyrano[2,3-c]pyridin-2-yl)-phenyl]-carbamic acid ethyl ester
[5-Hydroxy-2-(8-hydroxy-4-oxo-4H-pyrano[2,3-c]pyridin-2-yl)-phenyl]-carbamic acid ethyl ester

### c) 2-(4-Hydroxy-phenyl)-pyrano[3,2-c]pyridin-4-one

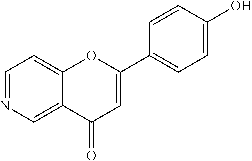 [SMILES: O=c1cc(-c2ccc(O)cc2)oc2ccncc12]

and its derivatives, including but not limited to:
2-(2,4-Dihydroxy-phenyl)-pyrano[3,2-c]pyridin-4-one
2-(3,4-Dihydroxy-phenyl)-pyrano[3,2-c]pyridin-4-one
5-Hydroxy-2-(4-hydroxy-phenyl)-pyrano[3,2-c]pyridin-4-one
7-Hydroxy-2-(4-hydroxy-phenyl)-pyrano[3,2-c]pyridin-4-one
8-Hydroxy-2-(4-hydroxy-phenyl)-pyrano[3,2-c]pyridin-4-one
[5-Hydroxy-2-(4-oxo-4H-pyrano[3,2-c]pyridin-2-yl)-phenyl]-carbamic acid ethyl ester
[3,5-Dihydroxy-2-(4-oxo-4H-pyrano[3,2-c]pyridin-2-yl)-phenyl]-carbamic acid ethyl ester
[4,5-Dihydroxy-2-(4-oxo-4H-pyrano[3,2-c]pyridin-2-yl)-phenyl]-carbamic acid ethyl ester
[5-Hydroxy-2-(5-hydroxy-4-oxo-4H-pyrano-3,2-c]pyridin-2-yl)-phenyl]-carbamic acid ethyl ester
[5-Hydroxy-2-(7-hydroxy-4-oxo-4H-pyrano[3,2-c]pyridin-2-yl)-phenyl]-carbamic acid ethyl ester
[5-Hydroxy-2-(8-hydroxy-4-oxo-4H-pyrano[3,2-c]pyridin-2-yl)-phenyl]-carbamic acid ethyl ester

### d) 2-(4-Hydroxy-phenyl)-pyrano[3,2-b]pyridin-4-one

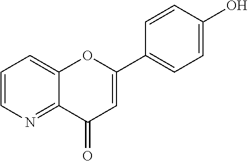 [SMILES: O=c1cc(-c2ccc(O)cc2)oc2cccnc12]

and its derivatives, including but not limited to:
2-(2,4-Dihydroxy-phenyl)-pyrano[3,2-b]pyridin-4-one
2-(3,4-Dihydroxy-phenyl)-pyrano[3,2-b]pyridin-4-one
6-Hydroxy-2-(4-hydroxy-phenyl)-pyrano[3,2-b]pyridin-4-one
7-Hydroxy-2-(4-hydroxy-phenyl)-pyrano[3,2-b]pyridin-4-one
8-Hydroxy-2-(4-hydroxy-phenyl)-pyrano[3,2-b]pyridin-4-one
[5-Hydroxy-2-(4-oxo-4H-pyrano[3,2-b]pyridin-2-yl)-phenyl]-carbamic acid ethyl ester
[3,5-Dihydroxy-2-(4-oxo-4H-pyrano[3,2-b]pyridin-2-yl)-phenyl]-carbamic acid ethyl ester
[4,5-Dihydroxy-2-(4-oxo-4H-pyrano[3,2-b]pyridin-2-yl)-phenyl]-carbamic acid ethyl ester
[5-Hydroxy-2-(6-hydroxy-4-oxo-4H-pyrano[3,2-b]pyridin-2-yl)-phenyl]-carbamic acid ethyl ester
[5-Hydroxy-2-(7-hydroxy-4-oxo-4H-pyrano[3,2-b]pyridin-2-yl)-phenyl]-carbamic acid ethyl ester
[5-Hydroxy-2-(8-hydroxy-4-oxo-4H-pyrano[3,2-b]pyridin-2-yl)-phenyl]-carbamic acid ethyl ester

### e) 7-(4-Hydroxy-phenyl)-pyrano[2,3-d]pyrimidin-5-one

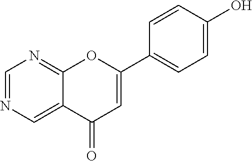 [SMILES: O=c1cc(-c2ccc(O)cc2)oc2ncncc12]

and its derivatives, including but not limited to:
7-(2,4-Dihydroxy-phenyl)-pyrano[2,3-d]pyrimidin-5-one
7-(3,4-Dihydroxy-phenyl)-pyrano[2,3-d]pyrimidin-5-one
4-Hydroxy-7-(4-hydroxy-phenyl)-pyrano[2,3-d]pyrimidin-5-one
2-Hydroxy-7-(4-hydroxy-phenyl)-pyrano[2,3-d]pyrimidin-5-one
[5-Hydroxy-2-(5-oxo-5H-pyrano[2,3-d]pyrimidin-7-yl)-phenyl]-carbamic acid ethyl ester
[3,5-Dihydroxy-2-(5-oxo-5H-pyrano[2,3-d]pyrimidin-7-yl)-phenyl]-carbamic acid ethyl ester
[4,5-Dihydroxy-2-(5-oxo-5H-pyrano[2,3-d]pyrimidin-7-yl)-phenyl]-carbamic acid ethyl ester
[5-Hydroxy-2-(4-hydroxy-5-oxo-5H-pyrano[2,3-d]pyrimidin-7-yl)-phenyl]-carbamic acid ethyl ester
[5-Hydroxy-2-(2-hydroxy-5-oxo-5H-pyrano[2,3-d]pyrimidin-7-yl)-phenyl]-carbamic acid ethyl ester

### f) 6-(4-Hydroxy-phenyl)-pyrano[3,2-d]pyrimidin-8-one

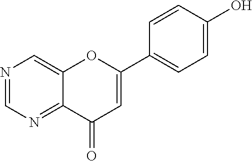 [SMILES: O=c1cc(-c2ccc(O)cc2)oc2cncnc12]

and its derivatives, including but not limited to:
6-(2,4-Dihydroxy-phenyl)-pyrano[3,2-d]pyrimidin-8-one
6-(3,4-Dihydroxy-phenyl)-pyrano[3,2-d]pyrimidin-8-one
2-Hydroxy-6-(4-hydroxy-phenyl)-pyrano[3,2-d]pyrimidin-8-one
4-Hydroxy-6-(4-hydroxy-phenyl)-pyrano[3,2-d]pyrimidin-8-one
[5-Hydroxy-2-(8-oxo-8H-pyrano[3,2-d]pyrimidin-6-yl)-phenyl]-carbamic acid ethyl ester
[3,5-Dihydroxy-2-(8-oxo-8H-pyrano[3,2-d]pyrimidin-6-yl)-phenyl]-carbamic acid ethyl ester
[4,5-Dihydroxy-2-(8-oxo-8H-pyrano[3,2-d]pyrimidin-6-yl)-phenyl]-carbamic acid ethyl ester
[5-Hydroxy-2-(2-hydroxy-8-oxo-8H-pyrano[3,2-d]pyrimidin-6-yl)-phenyl]-carbamic acid ethyl ester
[5-Hydroxy-2-(4-hydroxy-8-oxo-8H-pyrano[3,2-d]pyrimidin-6-yl)-phenyl]-carbamic acid ethyl ester

### g) 6-(4-Hydroxy-phenyl)-pyrano[2,3-b]pyrazin-8-one

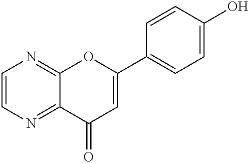 [SMILES: O=c1cc(-c2ccc(O)cc2)oc2nccnc12]

and its derivatives, including, but not limited to:
6-(2,4-Dihydroxy-phenyl)-pyrano[2,3-b]pyrazin-8-one
6-(3,4-Dihydroxy-phenyl)-pyrano[2,3-b]pyrazin-8-one
2-Hydroxy-6-(4-hydroxy-phenyl)-pyrano[2,3-b]pyrazin-8-one
3-Hydroxy-6-(4-hydroxy-phenyl)-pyrano[2,3-b]pyrazin-8-one
[5-Hydroxy-2-(8-oxo-8H-pyrano[2,3-b]pyrazin-6-yl)-phenyl]-carbamic acid ethyl ester
[3,5-Dihydroxy-2-(8-oxo-8H-pyrano[2,3-b]pyrazin-6-yl)-phenyl]-carbamic acid ethyl ester
[4,5-Dihydroxy-2-(8-oxo-8H-pyrano[2,3-b]pyrazin-6-yl)-phenyl]-carbamic acid ethyl ester
[5-Hydroxy-2-(2-hydroxy-8-oxo-8H-pyrano[2,3-b]pyrazin-6-yl)-phenyl]-carbamic acid ethyl ester
[5-Hydroxy-2-(3-hydroxy-8-oxo-8H-pyrano[2,3-b]pyrazin-6-yl)-phenyl]-carbamic acid ethyl ester.

The following compounds were obtained from commercially available sources (such as Indofine Chemical Company, Inc.): 2-(4-hydroxyphenyl)-chromen-4-one; 6-hydroxy-2-(4-hydroxyphenyl)-4H-chromen-4-one; 5,7-dihydroxy-2-phenyl-4H-chromen-4-one; 5-hydroxy-2-(4-hydroxyphenyl)-4H-chromen-4-one; 7-hydroxy-2-(4-hydroxyphenyl)-4H-chromen-4-one; 7-hydroxy-2-phenyl-4H-chromen-4-one; 5-hydroxy-2-phenyl-4H-chromen-4-one; 2-phenyl-4H-chromen-4-one; 2-(3-hydroxyphenyl)-4H-chromen-4-one; 7-methoxy-2-(4-hydroxyphenyl)-4H-chromen-4-one; 2-(4-hydroxy-3-methoxyphenyl)-4H-chromen-4-one; 5,7-dihydroxy-2-(4-hydroxyphenyl)-4H-chroman-4-one, 5,7-dihydroxy-3-(4-hydroxyphenyl)-4H-chromen-4-one; and 3,5,7-trihydroxy-2-(3,4-dihydroxyphenyl)-4H-chromen-4-one.

Abbreviations used herein denote the following compounds, reagents and substituents: acetic acid (AcOH); 2,2′-azobisisobutyronitrile (AIBN); N-bromosuccinimide (NBS); N-tert-butoxycarbonyl (Boc); t-butyldimethylsilyl (TBDMS); m-chloroperoxybenzoic acid (mCPBA); dimethylaminopyridine (DMAP); dichloromethane (DCM); dimethylformamide (DMF); dimethylsulfoxide (DMSO); ethanol (EtOH); ethyl acetate (EtOAc); 1-ethyl-3-(3-dimethylaminopropyl) carbodiimide (EDCl); 1-hydroxybenzotriazole (HOBt); iodomethane (MeI); lithium hexamethyldisilazide (LHMDS); methanol (MeOH); methoxymethyl (MOM); tetrahydrofuran (THF).

### Example 1

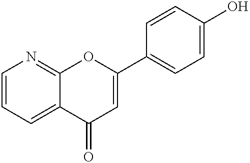 [SMILES: O=c1cc(-c2ccc(O)cc2)oc2ncccc12]

### 2-(4-Hydroxy-phenyl)-pyrano[2,3-b]pyridin-4-one

In a 500 mL dry round bottom flask with reflux condenser and magnetic stirrer was placed with 2-chloro-3-ethyl nicotinate (40.0 g, 215.5 mmol) in methanol (200 mL). CH₃ONa in methanol (25%, 65 mL, 301.7 mmol) was added slowly and the reaction mixture was refluxed for 16 h. The reaction was cooled to rt, quenched by addition of a saturated aqueous NH₄Cl solution. The aqueous mixture was extracted with ethyl acetate. The combined organic layers were washed well with water, brine, dried over Na₂SO₄ and concentrated to give 35 g of 2-methoxy-3-methyl nicotinate with 97% yield. Sodium hydride (60% in oil, 9.21 g, 230.3 mmol) was added to a dry 500 mL round bottom flask followed by 100 mL DMF. 4-Methoxyacetophenone (31.45 g, 209.44 mmol) in 50 mL dry DMF was added dropwise at 0° C. over 30 min. The reaction mixture was stirred for 1 h at rt. 2-Methoxynicotinic acid methyl ester (35 g, 209.44 mmol) was dissolved in 50 mL dry DMF and added slowly, keeping the temperature at 0° C. The mixture was stirred for 16 h at rt, then quenched by addition of a saturated aqueous NH₄Cl solution and diluted with water. The solid was filtered off, washed with water and dried to give 56.7 g diketo product in 95% yield.

The diketo compound (56.7 g, 198.9 mmol) was added to a 1 L round bottom flask together with pyridinium hydrochloride (345 g). The mixture was heated at 190° C. for 5 h. The reaction mixture was cooled to rt and diluted with water. The solid was isolated by filtration and purified by column chromatography using 5% methanol in CH₂Cl₂ to give 2-(4-hydroxy-phenyl)-pyrano[2,3-b]pyridin-4-one (23.25 g, 48.8%). MS (ES) m/z: 240.07 (M+1); ¹³C-NMR (DMSO-d₆): δ 178.2, 164.2, 161.8, 160.8, 153.9, 136.3, 129.2, 123.2, 121.8, 116.8, 116.75, 116.74, 105.7.

### Example 2

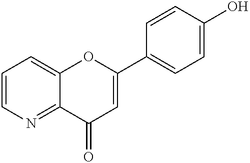 [SMILES: O=c1cc(-c2ccc(O)cc2)oc2cccnc12]

### 2-(4-Hydroxy-phenyl)-pyrano[3,2-b]pyridin-4-one

In a 500 mL round-bottom flask fitted with condenser and magnetic stirrer were placed MeOH (250 mL), 3-hydroxypyridine-2-carboxylic acid (10.0 g, 71.9 mmol) and concentrated H₂SO₄ (3 mL). The reaction mixture was heated at 64° C. for 24 hs. The reaction mixture was cooled to rt. The solvent was removed under reduced pressure; the residue was partitioned between EtOAc (150 mL) and water (20 mL). Solid sodium carbonate was added to adjust pH to 6. The organic layer was separated, dried over Na₂SO₄, concentrated to give 3.5 g of crude 3-hydroxypyridine-2-carboxylic acid methyl ester (32%).

In a 50 mL round-bottom flask fitted with magnetic stirrer were placed 3-hydroxypyridine-2-carboxylic acid methyl ester (3.5 g, 22.80 mmol), K₂CO₃ (3.46 g, 25.0 mmol), MeI (4.87 g, 34.3 mmol) and DMF (20 mL). The reaction mixture was stirred for 18 h at it under nitrogen. The reaction mixture was diluted with EtOAc (30 mL) and water (10 mL). The organic layer was separated and aqueous layer was extracted with EtOAc. The combined organic extracts were dried over Na₂SO₄ and concentrated to give the crude product. The crude product was purified by column chromatography using 30% EtOAc in hexane to give 2.1 g of 3-methoxypyridine-2-carboxylic acid methyl ester (54%).

In 100 mL round-bottom flask fitted with magnetic stirrer were placed the NaH (1.62 g of 60% suspension in mineral oil, 40 mmol) and the solution of t3-methoxypyridine-2-carboxylic acid methyl ester (3.5 g, 20 mmol) in anhydrous DMF (20 mL). The mixture was stirred for 15 min at it under N₂, then the solution of 4-methoxyacetophenone (3.3 g, 22 mmol) was added via syringe. The reaction mixture was stirred overnight at rt, then 10% aqueous solution of NaHSO₄ was used to adjust pH to 7. The organic layer was separated and aqueous layer was extracted with EtOAc. The combined organic extracts were dried over Na₂SO₄ and concentrated to give the crude product. The crude product was purified by column chromatography using 30% EtOAc in hexane to give 4.68 g of 1-(4-methoxyphenyl)-3-(3-methoxypyridin-2-yl) propane-1,3-dione (80%).

In 50 mL round-bottom flask fitted with magnetic stirrer were placed 1-(4-methoxyphenyl)-3-(3-methoxypyridin-2-yl) propane-1,3-dione (4.68 g, 16 mmol) and 45% aqueous solution of HBr (25 mL). The reaction mixture was refluxed for 3 h, then cooled down to rt. Solid NaHCO₃ was used to adjust pH to 7, followed by EtOAc (30 mL). The organic layer was separated and aqueous layer was extracted with EtOAc (2×30 mL). The combined organic extracts were dried over Na₂SO₄, concentrated to give the crude product, which was purified by column chromatography using 30% MeOH in EtOAc to give 125 mg of 2-(4-hydroxy-phenyl)-pyrano[3,2-b]pyridin-4-one (3.2%). MS (ES) m/z: 240.09 (M+1), and 149.06.

### Example 3

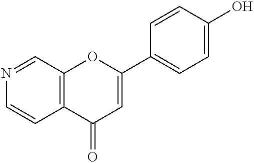 [SMILES: O=c1cc(-c2ccc(O)cc2)oc2cnccc12]

### 2-(4-Hydroxyphenyl)-pyrano[2,3-c]pyridin-4-one

A 50 mL flask was charged with 5.0 g (0.0354 mol) 3-fluoroisonicotinic acid and thionyl chloride (3.88 mL, 0.053 mol). The mixture was heated to reflux for 1 h, then the excess thionyl chloride was evaporated under vacuum. Anhydrous methanol was added to the residue and the mixture was heated to reflux for one hour. The reaction mixture was poured into sodium bicarbonate solution and pH was adjusted to 7.0. The mixture was extracted with EtOAc and the organic layer was dry over sodium sulfate. The organic solvent was evaporated yielding the product (4.80 g, 88%). A 50 mL dry flask was charged with methyl 3-fluoroisonicotinitate (3.50 g, 0.0227 mol), 4-methoxyacetophenone (3.60 g, 0.024 mol) and 10 mL dry DMF under nitrogen. Sodium hydride (1.82 g, 60% in oil) was added and the reaction was stirred for 30 min, then poured into ammonium chloride solution and extracted with EtOAc and dried over sodium sulfate. The solution was concentrated and the residue was pass through a column (EtOAc:hexane 1:3) to give the product (3.50 g, 54.0%). A 50 mL flask was charged with this product (0.5 g, 1.75 mmol) and pyridine hydrogen chloride (2.02 g, 17.5 mmol) and heat to 190° C. for 4 h. The mixture was poured into a sodium bicarbonate solution and the solid was collected by filtration, washed with EtOAc and methanol to give 2-(4-hydroxyphenyl)-pyrano[2,3-c]pyridin-4-one as a yellow product (0.36 g, 86%). MS (ES) m/z: 240.90 (M+1), 239.89 (M); Mp. 294-296° C.

### Example 4

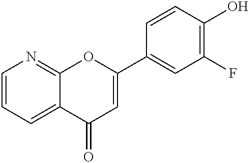 [SMILES: O=c1cc(-c2ccc(O)c(F)c2)oc2ncccc12]

### 2-(3-Fluoro-4-hydroxyphenyl)pyrano[2,3-b]pyridine-4-one

Methyl 2-methoxynicotinate was synthesized from ethyl 2-chloronicotinate with sodium methoxide as in Example 4. A 50 mL flask was charged with methyl 2-methoxynicotinate (2.50 g, 0.015 mol), 10 mL dry DMF and 60% NaH (0.745 g, 0.0186 mol) with magnetic stirring. 3′-Fluoro-4′-methoxyacetophenone (2.60 g, 0.0155 mol) in 6 mL anhydrous DMF was added over 5-10 min. After addition, the reaction mixture was stirred for 30 min. The mixture was poured into 50 mL NH₄Cl solution, the yellow solid was filtered and further washed with water and purified by column chromatography (hexane:EtOAc 4:1) to get (3.0 g, 66.4%) of product. A 50 mL flask was charged with this product (0.8 g, 2.64 mmol) and pyridine hydrogen chloride (3.04 g, 26.4 mmol) and heated to 190° C. for 4 h. The mixture was poured into sodium bicarbonate solution and the solid was collected by filtration, washed with EtOAc and MeOH and passed through a column (methanol:dichloromethane 1:4) to afford 400 mg of 2-(3-fluoro-4-hydroxyphenyl)pyrano[2,3-b]pyridine-4-one (59%). MS (ES) m/z: 257.85 (M); Mp. 267-268° C.

### Example 5

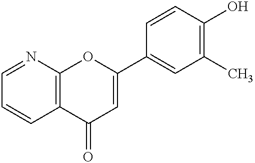 [SMILES: Cc1cc(-c2cc(=O)c3cccnc3o2)ccc1O]

### 2-(4-Hydroxy-3-methylphenyl)-4H-pyrano[2,3-b]pyridine-4-one

Methyl 2-methoxynicotinate was synthesized from ethyl 2-chloronicotinate with sodium methoxide as described in Example 4. A 100 mL dry flask was charged with 2-methylanisole (7.92 g, 65 mmol), acetyl chloride (5.1 mL, 71 mmol), aluminum chloride (9.45 g, 71 mmol) and 40 mL of anhydrous dichloromethane. The reaction mixture was kept at reflux for 2 h, then poured into 15 mL of HCl (3 N) and extracted with 100 mL ether. The organic layer was further washed with sodium bicarbonate to pH 6-7, then further washed with brine and dried over sodium sulfate. The solvent was evaporated and the residue was dried under high vacuum to yield the intermediate (10.0 g, 93.85%). A 100 mL dry flask was charged with methyl 2-methoxynicotinate (2.50 g, 15 mmol), 10 mL anhydrous DMF and NaH (0.9 g, 22.5 mmol, 60% in oil). The intermediate (2.58 g, 15.7 mmol) in 3 mL anhydrous DMF was added and the reaction was stirred for 2 hours. The mixture was poured into 120 mL of water with 3 mL AcOH. The yellow solid was further wash with water and passed through a column (hexane:EtOAc 3:1) to give the methoxy intermediate (3.4 g, 75.7%). A 50 mL flask was charged with the methoxy intermediate (1.0 g, 3.3 mmol) and pyridine hydrogen chloride (4.0 g, 33 mmol) and heated to 190° C. for 3 h. The mixture was poured into a sodium bicarbonate solution and the solid was collected by filtration, washed with EtOAc and MeOH (20 mL each) to give 2-(4-hydroxy-3-methylphenyl)-4H-pyrano[2,3-b]pyridine-4-one (0.58 g, 69.4%). MS (ES) m/z: 254.0 (M+1); Mp. 300-302° C.

### Example 6

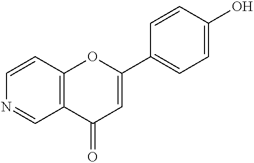 [SMILES: O=c1cc(-c2ccc(O)cc2)oc2ccncc12]

### 2-(4-Hydroxyphenyl)-4H-pyrano[3,2-c]pyridin-4-one

A solution of 4-chloropicolinic acid (3.0 g, 19.04 mmol) in EtOH (100 mL) was mixed with H₂SO₄ (conc., 5 mL) and was stirred at reflux for 48 h. The reaction mixture was cooled to rt and neutralized with NaOH (1 N) to adjust pH=8-9. The mixture was extract with dichloromethane (3×100 mL) and concentration to afforded ethyl 4-ethoxypicolinate (3.44 g, 93%).

To a solution of ethyl 4-ethoxypicolinate (3.44 g, 17.43 mmol) and 4-methoxy acetophenone (2.62 g, 17.43 mmol) in THF (100 mL) and DMSO (50 mL) was added NaH (1.4 g, 34.80 mmol). The resulting mixture was stirred at 95° C. for 6 h. The reaction mixture was cooled to rt and quenched with water (100 mL). The mixture was extract with EtOAc (3×150 mL) and concentration to a yellow solid. The solid was washed with hexanes to afford the diketone (3.6 g, 69%).

The diketone (1 g, 3.34 mmol) was mixed with pyridine hydrochloride (10 g). This mixture was stirred at 190° C. under N₂ for 12 h. The mixture was then diluted with EtOAc (30 mL) and poured into a beaker of 200 mL ice water. NaOH (1 N) was used to adjust pH=9. The solid was then filtered off and washed with water, hexanes, dichloromethane, EtOAc sequentially to afford the brownish solid 2-(4-hydroxyphenyl)-4H-pyrano[3,2-c]pyridin-4-one (0.39 g, 49%). MS (ES) m/z: 240.92 (M+1), 239.89 (M); Mp. 306-308° C.

### Example 7

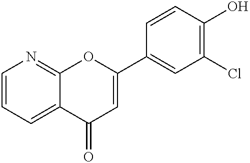 [SMILES: O=c1cc(-c2ccc(O)c(Cl)c2)oc2ncccc12]

### 2-(3-Chloro-4-hydroxyphenyl)-4H-pyrano[2,3-b]pyridine-4-one

Sodium methoxide (18 mL, 25 wt % in methanol) was added slowly to a solution of ethyl-2-chloronicotinate (11.134 g 60 mmol) in 60 mL anhydrous methanol. The reaction mixture was stirred under reflux for 15 h, then cooled to rt. Methanol was removed in vacuo. The residue was dissolved in EtOAc (200 mL) and saturated aqueous ammonium chloride (50 mL) was added. The organic layer was separated and dried over anhydrous Na₂SO₄. The solvent was removed to give ethyl-2-methoxynicotinate (8.58 g, 79%). Sodium hydride (60% in mineral oil, 0.48 g, 12 mmol) was dissolved in anhydrous DMF (10 mL). A solution of 3′-chloro-4′-methoxy acetophenone (1.85 g, 10 mmol) in anhydrous DMF (5 mL) was added drop-wise at 0° C. under nitrogen. The mixture was stirred at 0° C. for 5 min. and then at rt for 30 min. The mixture was cooled to 0° C. A solution of ethyl 2-methoxy nicotinate (1.81 g, 10 mmol) in anhydrous DMF (5 mL) was added slowly. The ice bath was removed and the mixture was stirring at it under nitrogen for 20 h. Water (20 mL) was added and the mixture was extracted with EtOAc (2×100 mL). The combined organic layers were washed with brine and dried over anhydrous Na₂SO₄. Removal of solvent gave a dark colored solid. Triturating with ether gave a yellow solid (1.64 g, 51%). The yellow solid (1.36 g, 4.21 mmol) and pyridinium hydrochloride (7.3 g, 63.2 mmol) were mixed together and stirred at 190° C. for 2 h, then cooled to rt. Water (100 mL) was added. The solid was separated by filtration, washed with water and dried under vacuum. The crude compound was purified by column chromatography (Silica Gel 230-400 mesh; 5% methanol in dichloromethane as an eluent to afford 2-(3-chloro-4-hydroxyphenyl)-4H-pyrano[2,3-b]pyridine-4-one (0.385 g, 33% yield) as yellow solid. MS (ES) m/z: 275.94+273. 92 (two isotopes of M); Mp. 259-262° C.

### Example 8

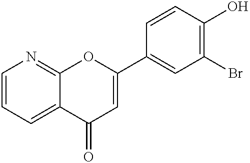 [SMILES: O=c1cc(-c2ccc(O)c(Br)c2)oc2ncccc12]

### 2-(3-Bromo-4-hydroxyphenyl)-4H-pyrano[2,3-b]pyridin-4-one

Sodium methoxide (18 mL, 25 wt % in methanol) was added slowly to a solution of ethyl-2-chloronicotinate (11.14 g 60 mmol) in anhydrous methanol (60 mL). The reaction mixture was stirred under reflux for 15 h, then cooled to rt. The methanol was removed in vacuo. The residue was dissolved in EtOAc (200 mL) and sat. ammonium chloride solution (50 mL) was added. The organic layer was separated and dried over anhydrous Na₂SO₄. Removal of solvent gave ethyl-2-methoxynicotinate (8.58 g, 79%) as yellow oil. Sodium hydride (0.21 g, 60% in mineral oil, 5.16 mmol) was mixed with anhydrous DMF (5 mL). A solution of 3′-bromo-4′-methoxyacetophenone (0.99 g, 4.3 mmol) in anhydrous DMF (3 mL) was added drop-wise at 0° C. under nitrogen. The mixture was stirred at 0° C. for 5 min. and then at it for 30 min. The mixture was cooled to 0° C. A solution of ethyl 2-methoxy nicotinate (1.81 g, 10 mmol) in anhydrous DMF (3 mL) was added slowly. The ice bath was removed and the stirring continued at it under nitrogen for 20 h. Water (20 mL) was added and the mixture was extracted with EtOAc (2×100 mL). The organic layer was washed with brine and dried over anhydrous Na₂SO₄. Removal of the solvent gave a dark solid. Triturating with ether gave a yellow solid (1.32 g, 84%). The solid (1.31 g, 3.6 mmol) and pyridinium hydrochloride (6.24 g, 54 mmol) were mixed together and stirred at 190° C. for 3 h, The reaction mixture was then cooled to rt, followed by the addition of water (200 mL). The solid was isolated by filtration, washed with water and dried under vacuum. The crude compound was purified by column chromatography (Silica Gel 230-400 mesh; 5:4:1 hexanes, EtOAc and methanol as an eluent) to give 2-(3-bromo-4-hydroxyphenyl)-4H-pyrano[2,3-b]pyridin-4-one (0.453 g, 40%) of as yellow solid. MS (ES) m/z: 317.84, 239.9; Mp. 267-272° C.

### Example 9

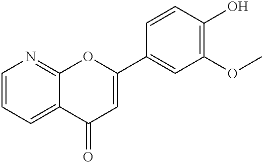 [SMILES: COc1cc(-c2cc(=O)c3cccnc3o2)ccc1O]

### 2-(4-Hydroxy-3-methoxyphenyl)-4H-pyrano[2,3-b]pyridine-4-one

A solution of ethyl 2-chloronicotinitate (6.0 g, 0.0323 mol) in anhydrous methanol (10 mL) at it was added sodium methoxide (10 mL, 25% in methanol). The reaction mixture was stirred for half hour then heated to reflux for one hour. The mixture was poured into water and extracted with ethyl acetate and the organic layer was washed with water until neutral, dried over sodium sulfate, and concentrated to give methyl 2-methoxynicotinitate (5.2 g, 96.3%).

A 100 mL dry flask was charged with acetovanillone (4.16 g, 0.025 mol) and anhydrous DMF (10 mL). Sodium hydride (1.05 g, 0.0263 mol, 60% in mineral oil) was added and the reaction mixture was stirred at it followed by the dropwise addition of benzyl bromide (3.1 mL, 0.0263 mol). The reaction was carried out at rt for 2 h, then poured into water. Ethyl acetate (150 mL) was used to extract out the compound and the organic layer was washed with water (2×100 mL), brine, dried over sodium sulfate, and concentrated to give the benzyl intermediate (6.21 g, 96%), which was subsequently used without further purification.

A 100 mL dry flask was charged with methyl 2-methoxynicotinitate (2.2 g, 0.0131 mol), the benzyl intermediate (3.37 g, 0.0131 mol) and anhydrous DMF (10 mL). Sodium hydride (0.524 g, 0.0131 mol, 60% in mineral oil) was added and the reaction mixture was stirred for 2 hours at rt. The reaction mixture was poured into water and extracted with ethyl acetate (150 mL). The organic layer was washed with water (2×100 mL), brine (100 mL), dried over sodium sulfate, and concentrated to give the intermediate (5.0 g, 97.6%). This intermediate (4.0 g, 0.0102 mol) and pyridine hydrochloride (12.0 g, 0.102 mol) were mixed and heated to 170-190° C. for 20 min. The reaction mixture was cooled and poured into water (100 mL). The mixture was extracted with ethyl acetate (3×200 mL), and the combined organic layers were washed with brine (3×100 mL), dried over sodium sulfate, and concentrated. The solid was further purified by refluxing with methanol (40 mL). The solution was cooled and filtered to yield 2-(4-hydroxy-3-methoxyphenyl)-4H-pyrano[2,3-b]pyridine-4-one (250 mg, 9.1%). MS (ES) m/z: 270.92, 269.91; Mp. 253-255° C.

### Example 10

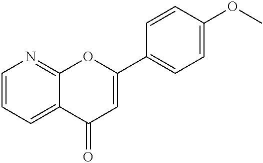 [SMILES: COc1ccc(-c2cc(=O)c3cccnc3o2)cc1]

### 2-(4-Methoxyphenyl)-4H-pyrano[2,3-b]pyridine-4-one

In a 500 mL dry round bottom flask with reflux condenser and magnetic stirrer was placed with 2-chloro-3-ethyl nicotinate (40.0 g, 215.5 mmol) in methanol (200 mL), and sodium methoxide (65 mL, 301.7 mmol, 25% in methanol) was added slowly and the reaction mixture was refluxed for 16 h. The reaction mixture was cooled to it and the reaction was quenched by addition of saturated aqueous NH₄Cl solution, followed by extraction with ethyl acetate. The combined organic layers were washed well with water, brine, dried over Na₂SO₄ and concentrated to give 2-methoxy-3-methyl nicotinate (35 g, 97%). To a dry 500 mL round bottom flask was added NaH (9.21 g 230.3 mmol, 60% in mineral oil) in DMF (100 mL). 4-Methoxyacetophenone (31.45 g, 209.44 mmol) in dry DMF (50 mL) was added dropwise at 0° C. over 30 minutes. The reaction mixture was stirred for 1 h at rt. Then 2-methoxynicotinic acid methyl ester (35 g, 209.44 mmol) dissolved in dry DMF (50 mL) was added slowly on cooling. The mixture was stirred for 16 h at rt. The reaction was quenched by addition of saturated NH₄Cl solution and diluted with water. The solid was filtered off, washed with water and dried to give the diketo product (56.7 g, 95%). Polyphosphoric acid (8.0 g) was heated at 90° C. and the diketo compound (1.0 g, 3.50 mmol) was added slowly and heated at 90° C. for 1 h. The reaction mixture was cooled to it and diluted with water. The solid was isolated by filtration, washed with water and dried to give 2-(4-methoxyphenyl)-4H-pyrano[2,3-b]pyridine-4-one (570 mg, 64%). MS (ES) m/z: 254.89 (M+1), 253.90 (M); Mp. 269-270° C.

### Example 11

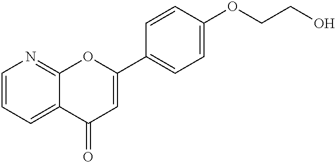 [SMILES: O=c1cc(-c2ccc(OCCO)cc2)oc2ncccc12]

### 2-(4-(2-Hydroxyethoxy)phenyl)-4H-pyrano[2,3-b]pyridine-4-one

In a 100 mL dry round bottom flask with reflux condenser and magnetic stirrer was placed 2-(4-hydroxy-phenyl)-pyrano[2,3-b]pyridin-4-one (1.0 g, 4.18 mmol) in EtOH (10 mL) and acetonitrile (50 mL). 2-Chloroethanol (2.05 g, 25.0 mmol) was added slowly and the reaction mixture was refluxed for 48 h. The reaction mixture was cooled to rt and concentrated under reduced pressure. The crude product was purified by column chromatography, using 2% MeOH in dichloromethane to afford 2-(4-(2-hydroxyethoxy)phenyl)-4H-pyrano[2,3-b]pyridine-4-one (380 mg, 32% yield). MS (ES) m/z: 284.94 (M+1), 283.95 (M); Mp. 157-159° C.

### Example 12

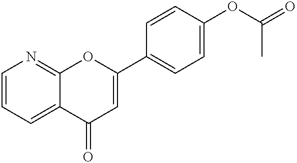 [SMILES: CC(=O)Oc1ccc(-c2cc(=O)c3cccnc3o2)cc1]

### 4-(4-Oxo-4H-pyrano[2,3-b]pyridine-2-yl)phenyl acetate

In a 50 mL round-bottomed flask fitted with condenser and magnetic stirrer were placed 2-(4-hydroxy-phenyl)-pyrano[2,3-b]pyridin-4-one (212 mg, 0.89 mmol), Ac₂O (99 mg, 0.97 mmol), and pyridine (5 mL). The reaction mixture was stirred for 24 h at room temperature. The reaction mixture was poured into water and extracted with EtOAc. The organic layer washed with water, dried and concentrated to afford 4-(4-oxo-4H-pyrano[2,3-b]pyridine-2-yl)phenyl acetate (240 mg, 96%). MS (ES) m/z: 282.89 (M+1), 281.92 (M); Mp. 167-169° C.

### Example 13

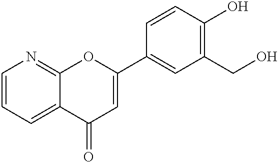 [SMILES: O=c1cc(-c2ccc(O)c(CO)c2)oc2ncccc12]

### 2-(4-Hydroxy-3-(hydroxymethyl)phenyl)-4H-pyrano[2,3′-b]pyridine-4-one

2-(4-Hydroxy-3-methylphenyl)-4H-pyrano[2,3-b]pyridine-4-one (0.91 g, 0.0036 mol), acetic anhydride (1.2 g, 0.0117 mol), DMAP (0.05 g) and triethylamine (10 mL) were added to a 50 ml flask and stirred overnight at rt. The solvent was removed and ethyl acetate (100 mL) was added and washed with water (80 mL), brine and dried over sodium sulfate. After the majority of the ethyl acetate was removed, hexane was added and the solid was isolated by filtration to give the acetylated intermediate (0.978 g, 92.0%).

The acetylated intermediate (0.50 g, 0.0017 mol) was dissolved into dry carbon tetrachloride (20 mL) and NBS (0.317 g, 0.00178 mol) was added. The reaction mixture was heated to reflux under a lamp for 3 h. After cooling the solid was filtered off and further washed with hot water to remove the succinimide. The methyl bromide was isolated by crystallization from DCM/Hexane (0.497 g, 78.2%).

The methyl bromide (0.49 g (0.0013 mol) and sodium acetate (1.07 g, 0.0131 mol) were mixed in acetic acid (20 mL) and heated to reflux for 16 hours. Acetic acid was removed and the residue was poured into water and extracted with ethyl acetate. The organic layer was washed with water, brine and dried in sodium sulfate. The solvent was removed and 0.50 g of the crude diacetylated compound was isolated. The diacetylated compound (0.50 g), potassium carbonate (0.45 g), and methanol (10 mL) were mixed and stirred for 3 hours. Acetic acid (2 mL) was added and the pH was adjusted to 5. The organic solvent was removed and the crude mixture was purified by column chromatography (DCM: MeOH 20:1) and then recrystallized to give 2-(4-hydroxy-3-(hydroxymethyl)phenyl)-4H-pyrano[3,2-b]pyridine-4-one (70 mg, 19.8%). MS (ES) m/z: 269.92 (M); Mp. 226-227° C.

### Example 14

### Inhibition of Tetra-Acetylated Histone H4 Binding Individual BET Bromodomains

Proteins were cloned and overexpressed with a N-terminal 6×His tag, then purified by nickel affinity followed by size exclusion chromatography. Briefly, E. coli BL21(DE3) cells were transformed with a recombinant expression vector encoding N-terminally Nickel affinity tagged bromodomains from Brd2, Brd3, Brd4. Cell cultures were incubated at 37° C. with shaking to the appropriate density and induced overnight with IPTG. The supernatant of lysed cells was loaded onto Ni-IDA column for purification. Eluted protein was pooled, concentrated and further purified by size exclusion chromatography. Fractions representing monomeric protein were pooled, concentrated, aliquoted, and frozen at −80° C. for use in subsequent experiments.

Binding of tetra-acetylated histone H4 and BET bromodomains was confirmed by a Time Resolved Fluorescence Resonance Energy Transfer (TR-FRET) method. N-terminally His-tagged bromodomains (200 nM) and biotinylated tetra-acetylated histone H4 peptide (25-50 nM, Millipore) were incubated in the presence of Europium Cryptate-labeled streptavidin (Cisbio Cat. #610SAKLB) and XL665-labeled monoclonal anti-His antibody (Cisbio Cat. #61HISXLB) in a white 96 well microtiter plate (Greiner). For inhibition assays, serially diluted test compound was added to these reactions in a 0.2% final concentration of DMSO. Final buffer concentrations were 30 mM HEPES pH 7.4, 30 mM NaCl, 0.3 mM CHAPS, 20 mM phosphate pH 7.0, 320 mM KF, 0.08% BSA). After 2 hours incubation at room temperature, the fluorescence by FRET was measured at 665 and 620 nm by a SynergyH4 plate reader (Biotek). Illustrative results with the first bromodomain of Brd4. Results are shown in Table 2. The binding inhibitory activity was shown by a decrease in 665 nm fluorescence relative to 620 nm. IC₅₀ values were determined from a dose response curve. Compounds with an IC₅₀ value less than 50 uM were deemed to be active.

TABLE 2
Inhibition of Binding of Tetra-acetylated Histone
H4 and Brd4 bromodomain 1 as Measured by FRET

| | FRET |
|---|---|
| | activity |
| Name | (<50 uM) |
| 2-(4-Hydroxy-phenyl)-pyrano[2,3-b]pyridin-4-one | Active |
| (Example 1) | |
| 2-(4-Hydroxy-phenyl)-pyrano[3,2-b]pyridin-4-one | Active |
| (Example 2) | |
| 2-(4-Hydroxyphenyl)-pyrano[2,3-c]pyridin-4-one | Not |
| (Example 3) | Active |
| 2-(3-Bromo-4-hydroxyphenyl)-4H-pyrano[2,3-b]pyridin-4- | Active |
| one (Example 8) | |
| 4-(4-Oxo-4H-pyrano[2,3-b]pyridine-2-yl)phenyl acetate | Not |
| (Example 12) | Active |

### Example 15

### Inhibition of c-Myc Expression in Cancer Cell Lines

MV4-11 cells (2.5×10⁴ cells) are plated in 96 well U-bottom plates with test compound or DMSO (0.1%), and incubated for 3 hours at 37° C. Cells are then harvested by centrifugation, lysed, and mRNA was isolated using the mRNA catcher plus kit (Invitrogen). Reverse transcription of the mRNA and duplex amplification of the c-myc and cyclophilin cDNAs are performed using the RNA Ultrasense kit (Invitrogen) and a ViiA7 real-time PCR machine (Applied Biosystems). IC₅₀ values are determined from a dose response curve. Compounds with an IC₅₀ value less than 30 uM are deemed to be active.

### Example 16

### Inhibition of Cell Proliferation in Cancer Cell Lines

MV4-11 cells: 96-well plates are seeded with 5×10⁴ cells per well of exponentially growing human AML MV-4-11 (CRL-9591) cells and immediately treated with two-fold dilutions of test compounds, ranging from 30 uM to 0.2 uM. Triplicate wells are used for each concentration, as well as a media only and three DMSO control wells. The cells and compounds are incubated at 37° C., 5% CO₂ for 72 hours before adding 20 uL of the CellTiter Aqueous One Solution (Promega) to each well and incubating at 37° C., 5% CO₂ for an additional 3-4 hours. The absorbance is taken at 490 nm in a spectrophotometer and the percentage of proliferation relative to DMSO-treated cells is calculated after correction from the blank well. IC₅₀ are calculated using the GraphPad Prism software. Compounds with an IC₅₀ value less than 30 uM are deemed to be active.

### Example 17

### Solubility Analysis

To evaluate the solubility of illustrative compounds of the invention, 1 mg of compound was added to 1 mL of PBS and sonicated for 1 hour at room temperature using the Branson 3210 Sonicator in triplicate and incubated in a water bath at 25° C. for 3 hrs. Samples were then centrifuged at 14,000 rpm for 6 minutes at room temperature. The supernatant was diluted with acetonitrile and was removed for analysis. Analysis was performed using HPLC-UV with 7-point standard curve to determine the concentration. The average concentration calculated was regarded as the solubility (μM). Table 3 shows the results of these experiments.

TABLE 3
Solubility Analysis

| | Solubility |
|---|---|
| Compound | (PBS) (μM) |
| 5,7-dihydroxy-2-(4-hydroxyphenyl)-4H-chomen-4-one | 3.37* |
| 2-(3-fluoro-4-hydroxyphenyl)pyrano[2,3-b]pyridine-4- | 194.50 |
| one (Example 4) | |
| 2-(4-hydroxyphenyl)-4H-pyrano[3,2-c]pyridine-4-one | 31.60 |
| (Example 6) | |
| 4′-hydroxyflavone | 5.04 |
| 2-(3-chloro-4-hydroxyphenyl)-4H-pyrano[2,3-b]pyridine- | 84.37 |
| 4-one (Example 7) | |
| 2-(3-bromo-4-hydroxyphenyl)-4H-pyrano[2,3-b]pyridin- | 52.59 |
| 4-one (Example 8) | |
| 2-(4-hydroxy-3-methoxyphenyl)-4H-pyrano[2,3- | 47.09 |
| b]pyridine-4-one (Example 9) | |
| 2-(4-(2-hydroxyethoxy)phenyl)-4H-pyrano[2,3- | 334.05 |
| b]pyridine-4-one (Example 11) | |
| 2-(4-hydroxy-3-(hydroxymethyl)phenyl)-4H-pyrano[2,3- | 48.54 |
| b]pyridin-4-one (Example 13) | |

*S. P. Ng et al., “Evaluation of the first-pass glucoronidation of select flavones in the gut by Caco2 monolayer model,” J. Pharm. Pharmaceut. Sci. 8(1): 1-9 (2005)

These experiments indicate that the solubility of representative compounds of the invention was significantly better than that of naturally occurring polyphenols, such as apigenin with a solubility of 3.27 μM. The poor bioavailability of naturally occurring polyphenols is partially attributed to poor solubility. As such, solubility is unlikely to affect the validity of any in vitro tests performed on the compounds of the invention, and formulation of these compounds for in vivo work should not be technically difficult to one skilled in the art. Accordingly, the compounds of the invention and pharmaceutically acceptable salts and hydrates thereof, are suitable for human use.

### Example 18

### Caco-2 Permeability

The Caco-2 cell drug transport model is widely used for screening compounds in drug discovery to assess intestinal transport and predict absorption rates. For example, the fraction of drug absorbed in human could be determined by in vivo human permeability or predicted by in vitro Caco-2 permeability; if compound permeability in Caco-2 cells reaches 13.3-18.1×10⁻⁶ cm/s, it is predicted that in vivo, permeability in humans would reach 2×10⁻⁴ cm/s, and the predicted fraction of drug absorbed would be >90%, which is defined as highly permeable. (D. Sun et al., “In vitro testing of drug absorption for drug ‘developability’ assessment: forming an interface between in vitro preclinical data and clinical outcome,” Curr. Opin. Drug Discov. Devel. 7(1):75-85 (2004). Therefore, in vitro absorption testing is a highly valuable tool for comparison of structural analogues for improved intestinal absorption, and to identify compounds within the decision-making process for clinical studies at early-stage drug discovery and development.

The method of B. Hai-Zhi et al., “High-throughput Caco-2 cell permeability screening by cassette dosing and sample pooling approaches using direct injection/on-line guard cartridge extraction/tandem mass spectrometry,” Rapid Communications in Mass Spectrometry 14:523-528 (2000) may be used with obvious modifications to someone skilled in the art. Table 4 shows the results of permeability of representative compounds of the invention in an in vitro Caco-2 intestinal transport model over time, and compared to resveratrol and 5,7-dihydroxy-2-(4-hydroxyphenyl)-4H-chomen-4-one (apigenin).

TABLE 4
Permeability Analysis

| | % Transported |
|---|---|
| Compound | Paap (cm/s) |
| Propanol | 1.01 × 10⁻⁵ |
| 7-methoxy-2-(4-hydroxyphenyl)-4H-chromen-4-one | 6.92 × 10⁻⁶ |
| 2-(4-hydroxy-phenyl)-pyrano[2,3-b]pyridin-4-one | 6.36 × 10⁻⁶ |
| (Example 1) | |
| 2-(4-hydroxy-phenyl)-pyrano[3,2-b]pyridin-4-one | 1.98 × 10⁻⁶ |
| (Example 2) | |
| 4′-hydroxyflavone | 4.31 × 10⁻⁶ |
| Resveratrol | 3.34 × 10⁻⁶ |
| 2-(3-fluoro-4-hydroxyphenyl)pyrano[2,3-b]pyridine- | 2.06 × 10⁻⁶ |
| 4-one (Example 4) | |
| 2-(4-hydroxy-3-methylphenyl)-4H-pyrano[2,3- | 5.96 × 10⁻⁷ |
| b]pyridine-4-one (Example 5) | |
| 2-(4-hydroxyphenyl)-4H-pyrano[3,2-c]pyridine-4- | 1.21 × 10⁻⁶ |
| one (Example 6) | |
| 2-(3-chloro-4-hydroxyphenyl)-4H-pyrano[2,3- | 1.76 × 10⁻⁶ |
| b]pyridine-4-one (Example 7) | |
| 2-(3-bromo-4-hydroxyphenyl)-4H-pyrano[2,3- | 9.59 × 10⁻⁷ |
| b]pyridin-4-one (Example 8) | |
| 5,7-dihydroxy-2-(4-hydroxyphenyl)-4H-chomen-4-one | 1.50 × 10⁻⁶ |
| 5,7-dihydroxy-2-phenyl-4H-chromen-4-one | 1.22 × 10⁻⁷ |

These experiments indicate that the permeability of representative compounds of the invention is equivalent to or greater than naturally occurring polyphenols, such as 5,7-dihydroxy-2-(4-hydroxyphenyl)-4H-chomen-4-one (apigenin) with a permeability of 1.50×10⁻⁶ or resveratrol with a permeability 3.34×10⁻⁶. Accordingly, the compounds of the invention and pharmaceutically acceptable salts and hydrates thereof, are potentially suitable for human use due to the permeability of intestinal cells to these compounds.

### Example 19

### Lipopolysaccharide (LPS) Stimulated Whole Blood Assay for Measuring TNFa and IL-6 Levels

Activation of monocytic cells by agonists of toll-like receptors such as bacterial lipopolysaccharide (LPS) results in production of key inflammatory mediators including IL-6 and TNFa. Such pathways are widely considered to be central to the pathophysiology of a range of auto-immune and inflammatory disorders. Compounds to be tested are diluted to give a range of appropriate concentrations and 1 μl of the dilution stocks is added to wells of a 96 plate. Following addition of whole blood (130 μL) the plates are incubated at 37 degrees (5% CO₂) for 30 min before the addition of 10 μl of 2.8 μg/mL lipopolysaccharides (LPS), diluted in complete RPMI 1640 (final concentration=200 ng/mL), to give a total volume of 140 uL per well. After further incubation for 24 hours at 37 degrees, 140 μL of PBS are added to each well. The plates are sealed, shaken for 10 minutes and then centrifuged (2500 rpm×10 min). 100 μL of the supernatant are removed and IL-6 and TNFa levels assayed by immunoassay (typically by MesoScale Discovery technology) either immediately or following storage at −20 degrees. BET inhibitors tested in this assay will inhibit the production of the key inflammatory mediator IL-6 and/or TNFa.

### Example 20

### In Vivo Mouse Endotoxemia Model Assay

High doses of Endotoxin (bacterial lipopolysaccharide) are administered to animals produce a profound shock syndrome including a strong inflammatory response, dysregulation of cardiovascular function, organ failure and ultimately mortality. This pattern of response is very similar to human sepsis and septic shock, where the body's response to a significant bacterial infection can be similarly life threatening. To test the compounds for use in the invention groups of Balb/c male mice are given a lethal dose of 15 mg/kg LPS by intraperitoneal injection. Ninety minutes later, animals are dosed intravenously with vehicle (20% cyclodextrin 1% ethanol in apyrogen water) or test compound (10 mg/kg). The survival of animals is evaluated at 4 days. BET inhibitors tested in the mouse endotoxemia model assay will result in a significant animal survival effect following intravenous administration.

### Example 21

### Growth Suppressive Activity Test Against Cancer Cells

Using RPMI 1640 medium (manufactured by SIGMA) supplemented with 10% fetal bovine serum, human promyelocytic leukemia-derived cell line HL-60, human acute lymphoblastic leukemia-derived cell line MOLT4, human Burkitt's lymphoma-derived cell line Daudi, and human multiple myeloma-derived cell line RPMI-8226 are each cultured at 37° C., 5% CO₂. In addition, using ISKOV medium (manufactured by SIGMA) supplemented with 10% fetal bovine serum, human chronic myeloid leukemia-derived cell line MV4-11 is cultured at 37° C., 5% CO₂. Moreover, using DMEM/F-12 medium (manufactured by SIGMA) supplemented with 10% fetal bovine serum, human lung cancer cell-derived cell line EBC-1, human hepatocellular cancer-derived cell line Kim-1, human colorectal cancer-derived cell line HCT-116, human prostate cancer-derived cell line PC-3, human ovarian cancer-derived cell line A2780, and human osteosarcoma-derived cell line Saos2 are each cultured at 37° C., 5% CO₂. These cells are plated on a 96 well plate, and cultured for 1 day. To each culture test compound diluted with the medium to a final concentration of 0.0003⁻¹⁰ μm (final DMSO concentration, 0.4%) is added. After culture for 3 more days, WST-8 (0.16 mg/mL) is added to the culture medium and the cells are cultured for 2 hr. The absorbance at 650 nm is subtracted from the absorbance at 450 nm. The growth suppressive activity is shown by a decrease rate of the absorbance of the group receiving test compound to that of the control group, and GI₅₀ value is determined from a dose-reaction curve plotting a decrease rate of the absorbance obtained by changing the compound concentrations.

This assay demonstrates that a compound that inhibits binding between acetylated histone, more specifically acetylated histone H4, and a bromodomain-containing protein, more specifically human-derived BET family protein BRD2, BRD3 or BRD4 can be used as an antitumor agent.

### Example 22

### HIV Tat-Mediated Transactivation Inhibition Assay

This assay evaluates inhibition of Tat-mediated transactivation by BET inhibitors that block the PCAF bromodomain interaction with HIV-1 Tat-AcK50. The effect is assessed by a microinjection study as described previously by Dorr et al. (EMBO J. 21; 2715-2723, 2002). In this microinjection assay, HeLa-Tat cells are grown on Cellocate coverslips and microinjected at room temperature with an automated injection system (Carl Zeiss). Samples are prepared as a 20 μl injection mix containing the LTR-luciferase (100 ng/ml) and CMV-GFP (50 ng/ml) constructs together with 5 mg/ml a chemical compound or pre-immune IgGs. Live cells are examined on a Zeiss Axiovert microscope to determine the number of GFP-positive cells. Four hours after injection, cells are washed in cold phosphate buffer and processed for luciferase assays (Promega). BET inhibitors tested in this assay will inhibit Tat-mediated transactivation by the PCAF BRD inhibitor.

### Example 23

### Whole Blood Assay IL-6 ELISA

Whole, fresh, heparinized blood is collected and diluted 1× in RPMI media+compounds and DMSO, in 1 mL volume total. Samples are incubated on a rotator, in the TC incubator, and treated for 1 h with compound and 3 h with 1 ug/mL LPS. Serum is harvested for ELISA analysis, and then RBCs are lysed with ammonium chloride, and lymphocytes are collected. Media is then harvested and ELISAs performed. The above experiment is performed in duplicate.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A method for inhibiting BET proteins in a mammal comprising administering a therapeutically effective amount of a compound of Formula I or a tautomer, stereoisomer, pharmaceutically acceptable salt, or hydrate thereof: 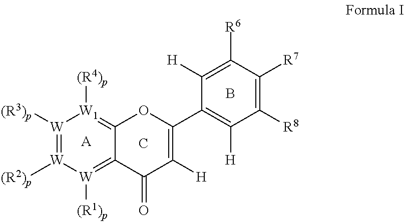
wherein:
R¹, R², R³, R⁴, R⁶, and R⁸, are each independently selected from (C₁-C₈)alkyl, (C₂-C₈)alkenyl, (C₂-C₈)alkynyl, aryl, heteroaryl, alkoxy, aryloxy, arylalkyl, amino, carbonyl, benzyl, phenyl, thioketone, hydrogen, hydroxyl, hydroxyalkyl, aminoalkyl, amides, carbamates, carboxy, cyano, cycloalkyl, ester, ether, formyl, halogen, heterocyclyl, haloalkyl, sulfonic acid [—SO₃H], phosphate, sulfonate, O-glucoronidate [the glucoronic (AKA glucuronic) acid conjugates], dicarboxylic acid, ketone, nitro, sulfide, sulfinyl, sulfonyl, sulfonamide and #STR55#, #STR66#, #STR77#, #STR88#, #STR99#, #STR100#,
R⁷ is selected from alkoxy, hydroxyl, hydroxyalkyl, ether, and ester, or
two adjacent substituents selected from R¹, R², R³, R⁶, R⁷, and R⁸, are connected in a 5 or 6-membered ring to form a bicyclic aryl or bicyclic heteroaryl when W₁ is N;
each W and/or W¹ is independently selected from C and N, wherein if W and/or W¹ is N, then p is 0 and if W and/or W¹ is C, then p is 1;
at least one W and/or W¹ is N;
wherein 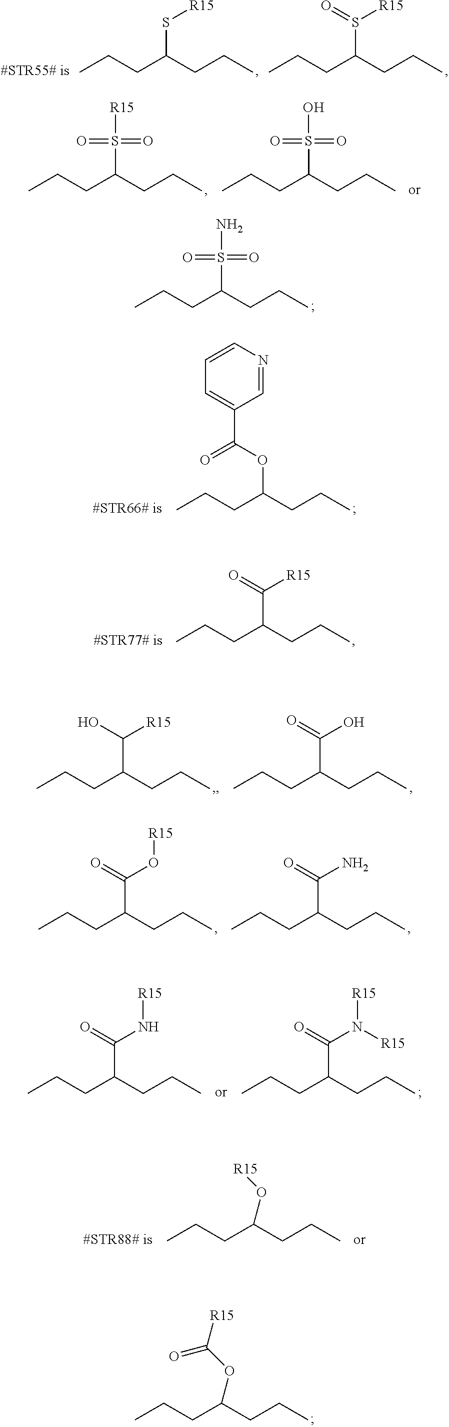 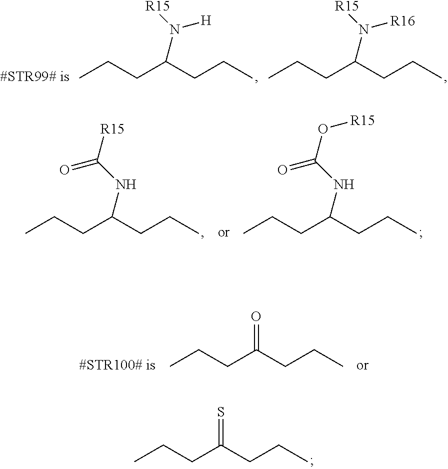
R¹⁵ and R¹⁶ are substituents independently selected from the group consisting of (C₁-C₈)alkyl, (C₂-C₈)alkenyl, (C₂-C₈)alkynyl, aryl, heteroaryl, alkoxy, aryloxy, benzyl, phenyl, carbonyl, hydrogen, hydroxyl [OH], acetyl, hydroxyalkyl, aminoalkyl, amides, carbamates, halogen, CF₃, CCl₃, sulfonic acid [—SO₃H], phosphate, or a derivative thereof, wherein said derivative is optionally substituted and optionally branched, and may have one or more of the C atoms replaced by S, N or O.

2. The method of claim 1, wherein the therapeutically effective amount of the compound of Formula I is administered with a pharmaceutically acceptable carrier in a pharmaceutically acceptable composition.

3. The method of claim 1, wherein the therapeutically effective amount of the compound of Formula I is sufficient to establish a concentration ranging from about 0.001 μM to about 100 μM in the mammal.

4. The method of claim 3, wherein the concentration ranges from about 1 μM to about 20 μM.

5. The method of claim 1, wherein the compound of Formula I is selected from:
2-(4-Hydroxy-phenyl)-pyrano[2,3-b]pyridin-4-one
2-(4-Hydroxy-phenyl)-pyrano[3,2-b]pyridin-4-one
2-(4-Hydroxyphenyl)-pyrano[2,3-c]pyridin-4-one
2-(3-Fluoro-4-hydroxyphenyl)pyrano[2,3-b]pyridine-4-one
2-(4-Hydroxy-3-methylphenyl)-4H-pyrano[2,3-b]pyridine-4-one
2-(4-Hydroxyphenyl)-4H-pyrano[3,2-c]pyridin-4-one
2-(3-Chloro-4-hydroxyphenyl)-4H-pyrano[2,3-b]pyridine-4-one
2-(3-Bromo-4-hydroxyphenyl)-4H-pyrano[2,3-b]pyridin-4-one
2-(4-Hydroxy-3-methoxyphenyl)-4H-pyrano[2,3-b]pyridine-4-one
2-(4-Methoxyphenyl)-4H-pyrano[2,3-b]pyridine-4-one
2-(4-(2-Hydroxyethoxy)phenyl)-4H-pyrano[2,3-b]pyridine-4-one
4-(4-Oxo-4H-pyrano[2,3-b]pyridine-2-yl)phenyl acetate
2-(4-Hydroxy-3-(hydroxymethyl)phenyl)-4H-pyrano[2,3]-b]pyridine-4-one
and stereoisomers, tautomers, pharmaceutically acceptable salts, and hydrates thereof.

6. The method of claim 5, wherein the therapeutically effective amount of the compound is administered with a pharmaceutically acceptable carrier in a pharmaceutically acceptable composition.

7. The method according to claim 1, wherein the method comprises treating a disease or disorder that is sensitive to a BET inhibitor.

8. The method of claim 1, wherein the disease or disorder is a cancer.

9. The method of claim 8, wherein the cancer is selected from the group consisting of cancers that exhibit c-myc overexpression, cancers that overexpress n-myc, cancers that that rely on the recruitment of p-TEFb to regulate activated oncogenes, Burkitt's lymphoma, acute myelogenous leukemia, multiple myeloma, aggressive human medulloblastoma, hematological, epithelial including lung, breast and colon carcinomas, midline carcinomas, and mesenchymal, hepatic, renal and neurological tumors.

10. The method of claim 8, wherein, the compound induces apoptosis in cancer cells by decreasing expression of the anti-apoptosis gene Bcl2.

11. The method of claim 8, wherein the compound of Formula I is administered in combination with another anti-cancer agent.

12. The method of claim 11, wherein the anti-cancer agent is selected from the group consisting of bortezomib, thalidomide, dexamethasone, 5-azacitidine, decitabine, vorinostat, and cyclophosphamide, a PI3K or mTOR inhibitor, rapamycin or a rapamycin analog, a gamma secretase inhibitor, an AMPK inducer, metformin, phenformin, an ornithine decarboxylase inhibitor, and difluoromethylornithine.

13. The method of claim 7, wherein the disease or disorder is an autoimmune or inflammatory disease.

14. The method of claim 7, wherein the disease or disorder is caused by bacterial or viral infection.

15. The method of claim 7, wherein the disease or disorder is AIDS.

16. The method of claim 7, wherein the disease or disorder is sepsis.
